(19) United States
(12) Patent Application Publication
(10) Pub. No.: US 2013/0331261 A1
(43) Pub. Date: Dec. 12, 2013

(54) PYRAZOLE CARBOXYLIC ACID AMIDES USEFUL FOR THE REDUCTION OF MYCOTOXIN CONTAMINATION IN PLANTS

(71) Applicants: Sebastian Hoffmann, Neuss (DE); Ruth Meissner, Leverkusen (DE); Pierre-Yves Coqueron, Lyon (FR); Philippe Desbordes, Lyon (FR)

(72) Inventors: Sebastian Hoffmann, Neuss (DE); Ruth Meissner, Leverkusen (DE); Pierre-Yves Coqueron, Lyon (FR); Philippe Desbordes, Lyon (FR)

(73) Assignee: BAYER INTELLECTUAL PROPERTY GMBH, Monheim (DE)

(21) Appl. No.: 13/990,636

(22) PCT Filed: Nov. 28, 2011
(86) PCT No.: PCT/EP2011/071167

§ 371 Date: Aug. 19, 2013

Related U.S. Application Data
(60) Provisional application No. 61/419,442, filed on Dec. 3, 2010.

(30) Foreign Application Priority Data
Dec. 1, 2010 (EP) 10193327.3

(51) Int. Cl. A01N 43/56; C07D 231/14

(52) U.S. Cl. CPC A01N 43/56; C07D 231/14
USPC 504/100; 514/406; 548/374.1

(57) ABSTRACT

The present invention relates to the novel use of pyrazole carboxylic acid amides, compositions comprising these compounds and their use in methods for the reduction of mycotoxin contamination in plants.

The present invention relates to the novel use of pyrazole carboxylic acid amides, compositions comprising these compounds and their use in methods for the reduction of mycotoxin contamination in plants.

Numerous fungi are serious pests of economically important agricultural crops. Further, crop contamination by fungal toxins is a major problem for agriculture throughout the world.

Mycotoxins, such as aflatoxins, ochratoxins, patulin, fumonisins, zearalenones, and trichothecenes, are toxic fungal metabolites, often found in agricultural products that are characterized by their ability to cause health problems for humans and vertebrates. They are produced for example by different Fusarium and Aspergillus, Penicillium and Alternaria species.

Aflatoxins are toxins produced by Aspergillus species that grow on several crops, in particular on maize or corn before and after harvest of the crop as well as during storage. The biosynthesis of aflatoxins involves a complex polyketide pathway starting with acetate and malonate. One important intermediate is sterigmatocystin and O-methylsterigmatocystin which are direct precursors of aflatoxins. Important producers of aflatoxins are Aspergillus flavus, most strains of Aspergillus parasiticus, Aspergillus nomius, Aspergillus bombycis, Aspergillus pseudotamarii, Aspergillus ochraceoroseus, Aspergillus rambelli, Emericella astellata, Emericella venezuelensis, Bipolaris spp., Chaetomium spp., Farrowia spp., and Monocillium spp., in particular Aspergillus flavus and Aspergillus parasiticus (Plant Breeding (1999), 118, pp 1-16). There are also additional Aspergillus species known. The group of aflatoxins consists of more than 20 different toxins, in particular aflatoxin B1, B2, G1 and G2, cyclopiazonic acid (CPA).

Ochratoxins are mycotoxins produced by some Aspergillus species and Penicilium species, like A. ochraceus, A. carbonarius or P. viridicatum, Examples for Ochratoxins are ochratoxin A, B, and C. Ochratoxin A is the most prevalent and relevant fungal toxin of this group.

Fumonisins are toxins produced by Fusarium (F.) species that grow on several crops, mainly corn, before and after harvest of the crop as well as during storage. The diseases, Fusarium kernel, ear and stalk rot of corn, is caused by Fusarium verticillioides, F. subglutinans, F. moniliforme, and F. proliferatum. The main mycotoxins of these species are the fumonisins, of which more than ten chemical forms have been isolated. Examples for fumonisins are FB1, FB2 and FB3. In addition the above mentioned Fusarium species of corn can also produce the mycotoxins moniliformin and beauvericin. In particular Fusarium verticillioides is mentioned as an important pathogen of corn, this Fusarium species produces as the main mycotoxin fumonisins of the B-type.

Trichothecenes are those mycotoxins of primary concern which can be found in Fusarium diseases of small grain cereals like wheat, barley, rye, triticale, rice, sorghum and oat. They are sesquiterpene epoxide mycotoxins produced by species of Fusarium, Trichothecium, and Myrothecium and act as potent inhibitors of eukaryotic protein synthesis.

Some of these trichothecene producing Fusarium species also infect corn or maize.

Examples of trichothecene mycotoxins include T-2 toxin, HT-2 toxin, isotrichodermol, DAS, 3-deacetylcalonectrin, 3,15-dideacetylcalonectrin, scirpentriol, neosolaniol; 15-acetyldeoxynivalenol, 3-acetyldeoxynivalenol, nivalenol, 4-acetylnivalenol (fusarenone-X), 4,15-diacetylnivalenol, 4,7,15-acetylnivalenol, and deoxynivalenol (hereinafter “DON”) and their various acetylated derivatives. The most common trichothecene in Fusarium head blight is DON produced for example by Fusarium graminearum and F. culmorum.

Another mycotoxin mainly produced by F. culmorum, F. graminearum and F. cerealis is zearalenone, a phenolic resorcyclic acid lactone that is primarily an estrogenic fungal metabolite.

Fusarium species that produce mycotoxins, such as fumonisins and trichothecenes, include F. acuminatum, F. crookwellense, F., verticillioides, F. culmorum, F. avenaceum, F. equiseti, F. moniliforme, F. graminearum (Gibberella zeae), F. lateritium, F. poae, F. sambucinum (G. pulicaris), F. proliferatum, F. subglutinans, F. sporotrichioides and other Fusarium species.

In contrast the species Microdochium nivale also a member of the so-called Fusarium complex is known to not produce any mycotoxins.

Both acute and chronic mycotoxicoses in farm animals and in humans have been associated with consumption of wheat, rye, barley, oats, rice and maize contaminated with Fusarium species that produce trichothecene mycotoxins. Experiments with chemically pure trichothecenes at low dosage levels have reproduced many of the features observed in moldy grain toxicoses in animals, including anemia and immunosuppression, haemorrage, emesis and feed refusal. Historical and epidemiological data from human populations indicate an association between certain disease epidemics and consumption of grain infected with Fusarium species that produce trichothecenes. In particular, outbreaks of a fatal disease known as alimentary toxic aleukia, which has occurred in Russia since the nineteenth century, have been associated with consumption of over-wintered grains contaminated with Fusarium species that produce the trichothecene T-2 toxin. In Japan, outbreaks of a similar disease called akakabi-byo or red mold disease have been associated with grain infected with Fusarium species that produce the trichothecene, DON. Trichothecenes were detected in the toxic grain samples responsible for recent human disease outbreaks in India and Japan. There exists, therefore, a need for agricultural methods for preventing, and crops having reduced levels of, mycotoxin contamination.

Further, mycotoxin-producing Fusarium species are destructive pathogens and attack a wide range of plant species. The acute phytotoxicity of mycotoxins and their occurrence in plant tissues also suggests that these mycotoxins play a role in the pathogenesis of Fusarium on plants. This implies that mycotoxins play a role in disease and, therefore, reducing their toxicity to the plant may also prevent or reduce disease in the plant. Further, reduction in disease levels may have the additional benefit of reducing mycotoxin contamination on the plant and particularly in grain where the plant is a cereal plant.

There is a need, therefore, to decrease the contamination by mycotoxins of plants and plant material before and/or after harvest and/or during storage.

N-[2-(phenyl)ethyl]-carboxamide derivatives and their use as fungicides are described in WO-A 2008/148570 and WO-A 2010/000612. Pyrazole-4-carboxylic acid amide derivatives and their use as pest-controlling agents are described in JP-2001-342179. Similar compounds are also known in other fields of technology, for example, the use of pyrazole-amides and sulfonamides as pain therapeutics is described in WO-A 2003/037274.

Therefore the problem to be solved by the present invention is to provide compounds which lead by their application on plants and/or plant material to a reduction in mycotoxins in all plant and plant material.

Accordingly, the present invention provides a method of reducing mycotoxin contamination in plants and/or any plant material and/or plant propagation material comprising applying to the plant or plant propagation material an effective amount of a compound of formula (I):

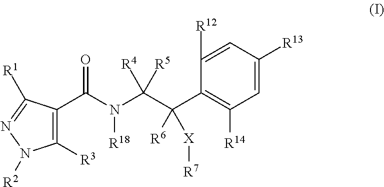

### Wherein

R¹ is halogenomethyl;
C₁-C₄-alkyl, C₁-C₄-halogenoalkyl, C₁-C₄-alkoxy-C₁-C₄alkyl or halogenoalkoxy-C₁-C₄-alkyl; and
R³ is hydrogen, halogen, methyl or cyano;
R⁴, R⁵ and R⁶ independently of each other stand for hydrogen, halogen, nitro, C₁-C₆-alkyl, which is unsubstituted or substituted by one or more substituents R⁸, C₃-C₆-alkyl, which is unsubstituted or substituted by one or more substituents R⁸, C₂-C₆-alkenyl, which is unsubstituted or substituted by one or more substituents R⁸, C₂-C₆-alkynyl, which is unsubstituted or substituted by one or more substituents R⁸;
   or R⁴ and R⁵ together are a C₂-C₅-alkylene group, which is unsubstituted or substituted by one or more C₁-C₆-alkyl groups:
X is oxygen, sulfur, —N(R¹⁰)— or —N(R¹¹)—O—;
R¹⁰ and R¹¹ independently of each other stand for hydrogen or C₁-C₆-alkyl;
R⁷ stands for C₁-C₆-alkyl, which is unsubstituted or substituted by one or more substituents R⁹, C₃-C₆-cycloalkyl, which is unsubstituted or substituted by one or more substituents R⁹, C₂-C₆-alkenyl, which is unsubstituted or substituted by one or more substituents R⁹, C₂-C₆-alkynyl, which is unsubstituted or substituted by one or more substituents R⁹;
R¹² stands for halogen, C₁-C₆-halogenoalkoxy, C₁-C₆-halogenoalkylthio, cyano, nitro, —C(R^{a})═N(OR^{b}), C₁-C₆-alkyl, which is unsubstituted or substituted by one or more substituents R¹⁵, C₃-C₆-cycloalkyl, which is unsubstituted or substituted by one or more substituents R¹⁵, C₆-C₁₄-bicycloalkyl, which is unsubstituted or substituted by one or more substituents R¹⁵, C₂-C₆-alkenyl, which is unsubstituted or substituted by one or more substituents R¹⁵, C₂-C₆-alkynyl, which is unsubstituted or substituted by one or more substituents R¹⁵, phenyl, which is unsubstituted or substituted by one or more substituents R¹⁵ phenoxy, which is unsubstituted or substituted by one or more substituents R¹⁵; or pyridinyloxy, which is unsubstituted or substituted by one or more substituents R¹⁵;
R¹³ stands for hydrogen, halogen, C₁-C₆-halogenoalkoxy, C₁-C₆-halogenoalkylthio, cyano, nitro, —C(R^{c})═N(OR^{d}), C₁-C₆-alkyl, which is unsubstituted or substituted by one or more substituents R¹⁶, C₃-C₆-cycloalkyl, which is unsubstituted or substituted by one or mire substituents R¹⁶, C₆-C₁₄-bicycloalkyl, which is unsubstituted or substituted by one or more substituents R¹⁶, C₆-C₁₄-alkenyl, which is unsubstituted or substituted by one or more substituents R¹⁶, C₂-C₆-alkynyl, which is unsubstituted or substituted by one or more substituents R¹⁶, phenyl, which is unsubstituted or substituted by one or more substituents R¹⁶, phenoxy, which is unsubstituted or substituted by one or more substituents R¹⁶ or pyridinyloy, which is unsubstituted or substituted by one or more substituents R¹⁶;
R¹⁴ stands for hydrogen, halogen, C₁-C₆-halogenoalkoxy, C₁-C₆-halogenoalkylthio, cyano, nitro, —C(R^{e})═N(OR^{f}), C₁-C₆-alkyl, which is unsubstituted or substituted by one or more substituents R¹⁷. C₃-C₆-cycloalkyl, which is unsubstituted or substituted by one or more substituents R¹⁷, C₆-C₁₄-bicycloalkyl, which is unsubstituted or substituted by one or more substituents R¹⁷, C₂-C₆-alkenyl, which is unsubstituted or substituted by one or more substituents R¹⁷, C₂-C₆-alkynyl, which is unsubstituted or substituted by one or more substituents R¹⁷, phenyl, which is unsubstituted or substituted by one or more substituents R¹⁷, phenoxy, which is unsubstituted or substituted by one or more substituents R¹⁷ or pyridinyloxy, which is unsubstituted or substituted by one or more substituents R¹⁷;
   each R⁸, R⁹, R¹⁵, R¹⁶ and R¹⁷ is independently of each other halogen, nitro, C₁-C₆-alkoxy, C₁-C₆-halogenoalkoxy, C₁-C₆-alkylthio, C₁-C₆-halogenoalkylthio, C₃-C₆-alkenyloxy, C₃-C₆-alkynyloxy or —C(R^{g})═N(OR^{h});
   each R^{a}, R^{c} R^{e} and R^{g} is independently of each other hydrogen or C₁-C₆-alkyl;
   each R^{b}, R^{d} R^{f} and R^{h} is independently of each other C₁-C₆-alkyl;

R¹⁸ is hydrogen or C₃-C₇-cycloalkyl;

and tautomers/isomers/enantiomers of these compounds.

The alkyl groups occurring in the definitions of the substituents can be straight-chain or branched and are, for example, methyl, ethyl, n-propyl, n-butyl, n-pentyl, n-hexyl, iso-propyl, see-butyl, iso-butyl or tert-butyl.

Alkoxy, alkenyl and alkynyl radicals are derived from the alkyl radicals mentioned. The alkenyl and alkynyl groups can be mono- or di-unsaturated.

The cycloalkyl groups occurring in the definitions of the substituents are, for example, cyclopropyl, cyclobutyl, cyclopentyl, cyclohexyl or cycloheptyl.

The bicycloalkyl groups occurring in the definitions of the substituents are, depending on the ring size, bicyclo[2.1.1]hexane, bicyclo[2.2.1]heptane, bicyclo[2.2.2]octane, bicyclo[3.2.1]octane, bicyclo[3.2.2]nonane, bicyclo[4.2.2]decane, bicyclo[4.3.2]undecane, adamantane and the like.

Halogen is generally fluorine, chlorine, bromine or iodine, preferably fluorine, bromine or chlorine. This also applies, correspondingly, to halogen in combination with other meanings, such as halogenoalkyl or halogenoalkoxy.

Halogenoalkyl groups preferably have a chain length of from 1 to 4 carbon atoms. Halogenoalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloromethyl, difluorochloromethyl, difluoromethyl, trifluoromethyl and dichlorofluoromethyl.

Suitable halogenoalkenyl groups are alkenyl groups which are mono- or polysubstituted by halogen, halogen being fluorine, chlorine, bromine and iodine and in particular fluorine and chlorine, for example 2,2-difluoro-1-methylvinyl, 3-fluoropropenyl, 3-chloropropenyl, 3-bromopropenyl, 2,3,3-trifluoropropenyl, 2,3,3-trichloropropenyl and 4,4,4-trifluorobut-2-en-1-yl.

Suitable halogenoalkynyl groups are, for example, alkynyl groups which are mono- or polysubstituted by halogen, halogen being bromine, iodine and in particular fluorine and chlorine, for example 3-fluoropropynyl, 3-chloropropynyl, 3-bromopropynyl, 3,3,3-trifluoropropynyl and 4,4,4-trifluorobut-2-yn-1-yl.

Alkoxy is, for example, methoxy, ethoxy, propoxy, iso-propoxy, n-butoxy, iso-butoxy, sec-butoxy and tert-butoxy; preferably methoxy and ethoxy.

Halogenoalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy and 2,2,2-trichloroethoxy; preferably difluoromethoxy, 2-chloroethoxy and trifluoromethoxy.

Alkylthio is, for example, methylthio, ethylthio, propylthio, iso-propylthio, n-butylthio, iso-butylthio, sec-butylthio or tert-butylthio, preferably methylthio and ethylthio.

Alkoxyalkyl is, for example, methoxymethyl, methoxyethyl, ethoxymethyl, ethoxyethyl, n-propoxymethyl, n-propoxyethyl, iso-propoxymethyl or iso-propoxyethyl.

In the context of the present invention “substituted by one or more substituents” in the definition of substituents R⁴, R⁵, R⁶, R⁷, R¹², R¹³ and R¹⁴, means typically, depending on the chemical structure of substituents R⁴, R⁵, R⁶, R⁷, R¹², R¹³ and R¹⁴, monosubstituted to nine-times substituted, preferably monosubstituted to five-times substituted, more preferably mono-, double- or triple-substituted.

The compounds of the formula I, wherein R¹⁸ is hydrogen, may occur in different tautomeric forms. For example, compounds of formula I exist in the tautomeric forms I_{I} and I_{N}:

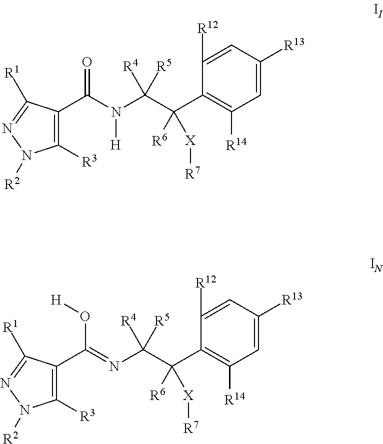

The invention covers all those tautomeric forms and mixtures thereof.

Preferably R¹⁸ is hydrogen.

In further preferred compounds of formula I, R¹ is CF₃, CF₂H or CFH₂, preferably CF₂H or CF₃, more preferably CF₂H; R² is C₁-C₄-alkyl, preferably methyl; and R¹ is hydrogen or halogen, preferably hydrogen or chlorine or fluorine. In one embodiment of the invention. R¹ is CF₂H; R² is methyl and R³ is hydrogen, preferably methyl; and R³ is hydrogen or halogen, preferably hydrogen or chlorine or fluorine. In one embodiment of the invention. R¹ is CF₂H; R² is methyl and R³ is chlorine. In one embodiment of the invention, R¹ is CF₂H; R² is methyl and R³ is fluorine. In one embodiment of the invention, R¹ is CF₃; R² is methyl and R³ is chlorine. In one embodiment of the invention, R¹ is CF₃; R² is methyl and R³ is fluorine.

In preferred compounds of formula I, R⁴ is selected from hydrogen, halogen, nitro, C₁-C₆-alkyl, which is unsubstituted or substituted by one or more substituents R⁸, C₃-C₆-cycloalkyl, which is unsubstituted or substituted by one or more substituents R⁸, C₂-C₆-alkenyl, which is unsubstituted or substituted by one or more substituents R⁸, C₂-C₆-alkynyl, which is unsubstituted or substituted by one or more substituents R⁸.

In further preferred compounds of formula I, R⁴ is hydrogen or C₁-C₆-alkyl, which is unsubstituted or substituted by one or more substituents R⁸.

In further preferred compounds of formula I, R⁴ is hydrogen, C₁-C₆-alkyl or C₁-C₆-halogenoalkyl.

In further preferred compounds of formula I, R⁴ is hydrogen or C₁-C₆-alkyl.

In further preferred compounds of formula I R⁴ is hydrogen or methyl.

In further preferred compounds of formula I, R⁴ is hydrogen.

In further preferred compounds of formula I, R⁴ is methyl.

In further preferred compounds of formula I, R⁴ is selected from hydrogen, halogen, nitro, C₁-C₆-alkyl, which is unsubstituted or substituted by one or more substituents R⁸, C₂-C₆-cycloalkyl, which is unsubstituted or substituted by one or more substituents R⁸, C₂-C₆-alkenyl, which is unsubstituted or substituted by one or more substituents R⁸, C₂-C₆-alkynyl, which is unsubstituted or substituted by one or more substituents R⁸.

In further preferred compounds of formula I, R⁴ is C₁-C₆-alkyl, which is unsubstituted or substituted by one or more substituents R⁸.

In further preferred compounds of formula I, R⁴ is C₁-C₆-alkyl or C₁-C₆-halogenoalkyl.

In further preferred compounds of formula I, R⁴ is C₁-C₆-alkyl.

In further preferred compounds of formula I, R⁴ is C₁-C₆-halogenoalkyl, preferably CF₃, CF₂H or CH₂F.

In preferred compounds of formula I, R⁵ and R⁶ independently of each other stand for hydrogen, halogen, nitro, C₁-C₆-alkyl, which is unsubstituted or substituted by one or more substituents R⁸, C₃-C₆-cycloalkyl, which is unsubstituted or substituted by one or more substituents R⁸, C₂-C₆-alkenyl, which is unsubstituted or substituted by one or more substituents R⁸, C₂-C₆-alkynyl, which is unsubstituted or substituted by one or more substituents R⁸.

In further preferred compounds of formula I, R⁵ and R⁶ independently of each other stand for hydrogen or C₁-C₆-alkyl.

In further preferred compounds of formula I, R⁵ and R⁶ are both hydrogen.

In preferred compounds of formula I, R⁸ stands for halogen, C₁-C₆-alkoxy, C₁-C₆-halogenoalkoxy, C₁-C₆-alkylthio or C₁-C₆-halogenoalkylthio.

In further preferred compounds of formula I, R⁸ stands for halogen or C₁-C₆-alkoxy.

In preferred compounds of formula I, X is oxygen. In further preferred compounds of formula I, X is sulfur. In further preferred compounds of formula I, X is —N(R¹⁰)—. In further preferred compounds of formula I, X is —N(R¹¹)—O—.

In preferred compounds R¹⁰ is hydrogen or methyl.

In preferred compounds R¹¹ is hydrogen or methyl. In one embodiment of the invention R¹¹ is hydrogen.

In preferred compounds of formula I, R⁷ stands for C₁-C₆-alkyl, which is unsubstituted or substituted by one or more substituents R⁹, C₂-C₆-alkenyl, which is unsubstituted or substituted by one or more substituents R⁹ or C₂-C₆-alkynyl, which is unsubstituted or substituted by one or more substituents R⁹.

In further preferred compounds of formula I, R⁷ stands for C₁-C₆-alkyl, C₂-C₆-alkenyl or C₂-C₆-alkynyl.

In further preferred compounds of formula I, R⁷ stands for C₁-C₆-alkyl, preferably methyl.

In preferred compounds of formula I, R⁹ stands for halogen, C₁-C₆-alkoxy, C₁-C₆-halogenoalkoxy, C₁-C₆-alkylthio or C₁-C₆-halogenoalkylthio.

In further preferred compounds of formula I, R⁹ stands for halogen or C₁-C₆-alkoxy.

In preferred compounds, R¹² stands for halogen, C₁-C₆-halogenoalkoxy, C₁-C₆-halogenoalkylthio, cyano, nitro, —C(R^{a})═N(OR^{b}), C₁-C₆-alkyl, which is unsubstituted or substituted by one or more substituents R¹⁵, C₃-C₆-cycloalkyl, which is unsubstituted or substituted by one or more substituents R¹⁵, C₆-C₁₄-bicycloalkyl, which is unsubstituted or substituted by one or more substituents R¹⁵, C₂-C₆-alkenyl, which is unsubstituted or substituted by one or more substituents R¹⁵, C₁-C₆-alkynyl, which is unsubstituted or substituted by one or more substituents R¹⁵, phenyl, which is unsubstituted or substituted by one or more substituents R¹⁵, phenoxy, which is unsubstituted or substituted by one or more substituents R¹⁵ or pyridinyloxy, which is unsubstituted or substituted by one or more substituents R¹⁵;
R¹³ stands for halogen, C₁-C₆-halogenoalkoxy, C₁-C₆-halogenoalkylthio, cyano, nitro, —C(R^{c})═N(OR^{d}), C₁-C₆-alkyl, which is unsubstituted or substituted by one or more substituents R¹⁶, C₃-C₆-cycloalkyl, which is unsubstituted or substituted by one or more substituents R¹⁶, C₆-C₁₄-bicycloalkyl, which is unsubstituted or substituted by one or more substituents R¹⁶, C₂-C₆-alkenyl, which is unsubstituted or substituted by one or more substituents R¹⁶, C₂-C₆-alkynyl, which is unsubstituted or substituted by one or more substituents R¹⁶, phenyl, which is unsubstituted or substituted by one or more substituents R¹⁶, phenoxy, which is unsubstituted or substituted by one or more substituents R¹⁶ or pyridinyloxy, which is unsubstituted or substituted by one or more substituents R¹⁶;
and R¹⁴ stands for hydrogen, halogen, C₁-C₆-halogenoalkoxy, C₁-C₆-halogenoalkylthio, cyano, nitro, —C(R^{c})═N(OR^{f}), C₁-C₆-alkyl, which is unsubstituted or substituted by one or more substituents R¹⁷, C₃-C₆-cycloalkyl, which is unsubstituted or substituted by one or more substituents R¹⁷, C₆-C₁₄-bicycloalkyl, which is unsubstituted or substituted by one or more substituents R¹⁷, C₂-C₆-alkenyl, which is unsubstituted or substituted by one or more substituents R¹⁷, C₂-C₆-alkynyl, which is unsubstituted or substituted by one or more substituents R¹⁷, phenyl, which is unsubstituted or substituted by one or more substituents R¹⁷, phenoxy, which is unsubstituted or substituted by one or more substituents R¹⁷ or pyridinyloxy, which is unsubstituted or substituted by one or more substituents R¹⁷.

In preferred compounds
R¹² and R¹³ independently of one another are halogen, cyano, C₁-C₆-alkyl, C₂-C₆-alkynyl, C₁-C₆-alkoxy, C₁-C₆-halogenoalkyl, C₁-C₆-halogenoalkoxy, —C(H)═N(O—C₁-C₆-alkyl) or phenyl, which is unsubstituted or substituted by one or more halogens; and R¹⁴ is hydrogen, halogen, cyano, C₁-C₆-alkyl, C₂-C₆-alkynyl, C₁-C₆-alkoxy, C₁-C₆-halogenoalkyl, C₁-C₆-halogenoalkoxy, —C(H)═N(O—C₁-C₆-alkyl) or phenyl, which is unsubstituted or substituted by one or more halogens.

In further preferred compounds
R¹² and R¹³ independently of one another are halogen, cyano, C₂-C₆-alkynyl, C₁-C₆-halogenoalkyl, C₁-C₆-halogenoalkoxy, —C(H)═N(O—C₁-C₆-alkyl) or phenyl, which is substituted halogen; and R¹⁴ is hydrogen, halogen, cyano. C₂-C₆-alkynyl, C₁-C₆-halogenoalkyl, C₁-C₆-halogenoalkoxy, —C(H)═N(O—C₁-C₆-alkyl) or phenyl, which is substituted halogen.

In further preferred compounds
R¹² and R¹³ independently of one another are halogen, C₂-C₆-alkynyl, C₁-C₆-halogenoalkyl or —C(H)═N(O—C₁-C₆-alkyl); and R¹⁴ is hydrogen, halogen, C₂-C₆-alkynyl, C₁-C₆-halogenoalkyl or —C(H)═N(O—C₁-C₆-alkyl).

In further preferred compounds
R¹² and R¹³ independently of one another are halogen or C₁-C₆-halogenoalkyl; and R¹⁴ is hydrogen, halogen or C₁-C₆-halogenoalkyl.

In further preferred compounds
R¹² and R¹³ independently of one another are halogen or C₁-C₆-halogenoalkyl, preferably halogen; and R¹⁴ is hydrogen.

In further preferred compounds

R¹², R¹³ and R¹⁴ independently of one another are halogen or C₁-C₆-halogenoalkyl, preferably halogen.

Further preferred compounds are listed in table 1:

TABLE 1

| Example No | Chemical Structure |
|---|---|
| 1 | 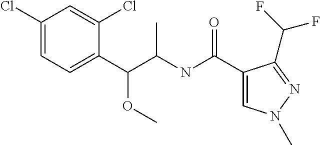 [SMILES: COC(c1ccc(Cl)cc1Cl)C(C)NC(=O)c1cn(C)nc1C(F)F] |
| 2 | 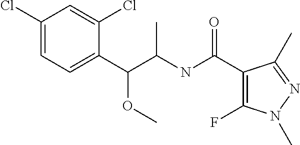 [SMILES: COC(c1ccc(Cl)cc1Cl)C(C)NC(=O)c1c(C)nn(C)c1F] |
| 3 | 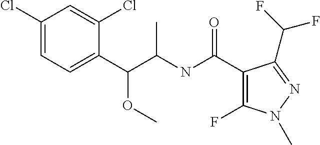 [SMILES: COC(c1ccc(Cl)cc1Cl)C(C)NC(=O)c1c(C(F)F)nn(C)c1F] |

Compounds of formula I may be prepared according to procedures described in WO-A 2008/148570 and WO-A 2010/000612.

As indicated above, it has now been found that the compounds of formula I are useful in reducing mycotoxin contamination when they are applied to a plant and/or any plant material and/or plant propagation material in an effective amount.

In a particular embodiment the compounds of formula I are useful in reducing mycotoxin contamination produced by fungi when they are applied to a plant and/or any plant material and/or plant propagation material in an effective amount.

The compounds of formula I are useful in reducing mycotoxin contamination when they are applied to a plant and/or any plant material and/or plant propagation material in an effective amount before and/or after harvest and/or during storage.

In a particular embodiment the compounds of formula I are useful in reducing mycotoxin contamination produced by fungi selected from the group of the following species: F. acuminatum, F. crookwellense, F. verticillioides, F. culmorum, F. avenaceum, F. equiseti, F. moniliforme, F. graminearum (Gibberella zeae), F. lateritium, F. poae, F. sambucinum (G. pulicaris), F. proliferatum, F. subglutinans and F. sporotrichioides, Aspergillus flavus, most strains of Aspergillus parasiticus and Aspergillus nomius, A. ochraceus, A. carbonarius or P. viridicatum when they are applied to a plant and/or any plant material and/or plant propagation material in an effective amount.

In a particular embodiment the compounds of formula I are useful in reducing mycotoxin contamination produced by fungi selected from the group of the following species: F. verticillioides, F. culmorum, F. moniliforme, F. graminearum (Gibberella zeae), F. proliferatum, Aspergillus flavus, most strains of Aspergillus parasiticus and Apergillus nomius, A. ochraceus, A. carbonarius when they are applied to a plant and/or any plant material and/or plant propagation material in an effective amount.

In a particular embodiment the compounds of formula I are useful in reducing mycotoxin contamination produced by fungi selected from the group of the following species: F. verticillioides, F. proliferatum, F. graminearum (Gibberella zeae), Aspergillus flavus, and Aspergillus parasiticus when they are applied to a plant and/or any plant material and/or plant propagation material in an effective amount.

In a particular embodiment the compounds of formula I are useful in reducing mycotoxin contamination produced by fungi selected from the group of the following species: F. verticillioides, F. proliferatum, F. graminearum when they are applied to a plant and/or any plant material and/or plant propagation material in an effective amount.

In a particular embodiment the compounds of formula I are useful in reducing mycotoxin contamination produced by fungi selected from the group of the following species: Aspergillus flavus, and Aspergillus parasiticus when they are applied to a plant and/or any plant material and/or plant propagation material in an effective amount.

In a particular embodiment the mycotoxins are selected from the following group: aflatoxins B1, B2, G1 and G2, ochratoxin A, B, C as well as T-2 toxin, HT-2 toxin, isotrichodermol, DAS, 3-deacetylcalonectrin, 3,15-dideacetylcalonectrin, scirpentriol, neosolaniol; zearalenone, 15-acetyldeoxynivalenol, nivalenol, 4-acetylnivalenol (fusarenone-X), 4,15-diacetylnivalenol, 4,7,15-acetylnivalenol, and deoxynivalenol (hereinafter “DON”) and their various acetylated derivatives as well as fumonisins of the B-type as FB1, FB2, FB3.

In a very particular embodiment the mycotoxins are selected from the following group: aflatoxins B1, B2, G and G2, zearalenone, deoxynivalenol (hereinafter “DON”) and their various acetylated derivatives as well as fumonisins of the B-type as FB1, FB2, FB3.

In a very particular embodiment the mycotoxins are selected from the following group: aflatoxins B1, B2, G1 and G2.

In a very particular embodiment the mycotoxins are selected from the following group: aflatoxins B1.

In a very particular embodiment the mycotoxins are selected from the following group: zearalenone, deoxynivalenol (hereinafter “DON”) and their various acetylated derivatives.

In a very particular embodiment the mycotoxins are selected from the following group: fumonisins of the B-type as FB1, FB2, FB3.

In a particular embodiment of the invention plant and/or plant material and/or plant propagation material has at least 10% less mycotoxin, more preferable at least 20% less mycotoxins, more preferable at least 40% less mycotoxins, more preferable at least 50% less mycotoxins more preferable at least 80% less mycotoxin contamination than plant or plant material which has not been treated.

In a particular embodiment of the invention plant and/or plant material and/or plant propagation material before and/or after harvest and/or during storage has at least 10% less mycotoxin, more preferable at least 20% less mycotoxins, more preferable at least 40% less mycotoxins, more preferable at least 50% less mycotoxins more preferable at least 80% less mycotoxin contamination than plant or plant material before and/or after harvest and/or during storage which has not been treated.

In a particular embodiment of the invention plant and/or plant material and/or plant propagation material before harvest has at least 10% less aflatoxins, more preferable at least 20% aflatoxin, more preferable at least 40% aflatoxins, more preferable at least 50% aflatoxins, more preferable at least 80% aflatoxin contamination than plant or plant material before harvest which has not been treated.

In a particular embodiment of the invention plant and/or plant material and/or plant propagation material after harvest has at least 10% less fumonisins, more preferable at least 20% fumonisins, more preferable at least 40% fumonisins, more preferable at least 50% fumonisins, more preferable at least 80% fumonisin contamination than plant or plant material after harvest which has not been treated.

In a particular embodiment of the invention plant and/or plant material and/or plant propagation material during storage has at least 10% less DON, more preferable at least 20% DON, more preferable at least 40% DON, more preferable at least 50% DON, more preferable at least 80% DON contamination than plant or plant during storage which has not been treated.

In a particular embodiment the compounds according to formula (I), especially those of table 1 can be combined with other active ingredients like fungicides, insecticides, herbicides, biological control agents.

In particular the fungicides are selected from the group comprising

(1) Inhibitors of the ergosterol biosynthesis, for example (1.1) aldimorph (1704-28-5), (1.2) azaconazole (60207-31-0), (1.3) bitertanol (55179-31-2), (1.4) bromuconazole (116255-48-2), (1.5) cyproconazole (113096-99-4), (1.6) diclobutrazole (75736-33-3), (1.7) difenoconazole (119446-68-3), (1.8) diniconazole (83657-24-3), (1.9) diniconazole-M (83657-18-5), (1.10) dodemorph (1593-77-7), (1.11) dodemorph acetate (31717-87-0), (1.12) epoxiconazole (106325-08-0), (1.13) etaconazole (60207-93-4), (1.14) fenarimol (60168-88-9), (1.15) fenbuconazole (114369-43-6), (1.16) fenhexamid (126833-17-8), (1.17) fenpropidin (67306-00-7), (1.18) fenpropimorph (67306-03-0), (1.19) fluquinconazole (136426-54-5), (1.20) flurprimidol (56425-91-3), (1.21) flusilazole (85509-19-9), (1.22) flutriafol (76674-21-0), (1.23) furconazole (112839-33-5), (1.24) furconazole-cis (112839-32-4), (1.25) hexaconazole (79983-71-4), (1.26) imazalil (60534-80-7), (1.27) imazalil sulfate (58594-72-2), (1.28) imibenconazole (86598-92-7), (1.29) ipconazole (125225-28-7), (1.30) metconazole (125116-23-6), (1.31) myclobutanil (88671-89-0), (1.32) naftifine (65472-88-0), (1.33) nuarimol (63284-71-9), (1.34) oxpoconazole (174212-12-5), (1.35) paclobutrazol (76738-62-0), (1.36) pefurazoate (101903-30-4), (1.37) penconazole (66246-88-6), (1.38) piperalin (3478-94-2), (1.39) prochloraz (67747-09-5), (1.40) propiconazole (60207-90-1), (1.41) prothioconazole (178928-70-6), (1.42) pyributicarb (88678-67-5), (1.43) pyrifenox (88283-41-4), (1.44) quinconazole (103970-75-8), (1.45) simeconazole (149508-90-7), (1.46) spiroxamine (118134-30-8), (1.47) tebuconazole (107534-96-3), (1.48) terbinafine (91161-71-6), (1.49) tetraconazole (112281-77-3), (1.50) triadimefon (43121-43-3), (1.51) triadimenol (89482-17-7), (1.52) tridemorph (81412-43-3), (1.53) triflumizole (68694-11-1), (1.54) triforine (26644-46-2), (1.55) triticonazole (131983-72-7), (1.56) uniconazole (83657-22-1), (1.57) uniconazole-p (83657-17-4), (1.58) viniconazole (77174-66-4), (1.59) voriconazole (137234-62-9), (1.60) 1-(4-chlorophenyl)-2-(1H-1,2,4-triazol-1-yl)cycloheptanol (129586-32-9), (1.61) methyl 1-(2,2-dimethyl-2,3-dihydro-1H-inden-1-yl)-1H-imidazole-5-carboxylate (110323-95-0), (1.62) N′-{5-(difluoromethyl)-2-methyl-4-[3-(trimethylsilyl)propoxy]phenyl}-N-ethyl-N-methylimidoformamide, (1.63) N-ethyl-N-methyl-N′-{2-methyl-5-(trifluoromethyl)-4-[3-(trimethylsilyl)propoxy]phenyl}imidoformamide and (1.64) O—[1-(4-methoxyphenoxy)-3,3-dimethylbutan-2-yl]1H-imidazole-1-carbothioate (111226-71-2).
(2) inhibitors of the respiratory chain at complex I or II, for example (2.1) bixafen (581809-46-3), (2.2) boscalid (188425-85-6), (2.3) carboxin (5234-68-4), (2.4) diflumetorim (130339-07-0), (2.5) fenfuram (24691-80-3), (2.6) fluopyram (658066-35-4), (2.7) flutolanil (66332-96-5), (2.8) fluxapyroxad (907204-31-3), (2.9) furametpyr (123572-88-3), (2.10) furmecyclox (60568-05-0), (2.11) isopyrazam (mixture of syn-epimeric racemate 1RS,4SR,9RS and anti-epimeric racemate 1RS,4SR,9SR) (881685-58-1), (2.12) isopyrazam (anti-epimeric racemate 1RS,4SR,9SR), (2.13) isopyrazam (anti-epimeric enantiomer 1R,4S,9S), (2.14) isopyrazam (anti-epimeric enantiomer 1S,4R,9R), (2.15) isopyrazam (syn epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil (55814-41-0), (2.19) oxycarboxin (5259-88-1), (2.20) penflufen (494793-67-8), (2.21) penthiopyrad (183675-82-3), (2.22) sedaxane (874967-67-6), (2.23) thifluzamide (130000-40-7), (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethyl)-N-[4-fluoro-2-(1,1,2,3,3,3-hexafluoropropoxy)phenyl]-1-methyl-1H-pyrazole-4-carboxamide, (2.27) N-[1-(2,4-dichlorophenyl)-1-methoxypropan-2-yl]-3-(difluoromethyl)-1-methyl-1H-pyrazole-4-carboxamide (1092400-95-7) (WO 2008148570), (2.28) 5,8-difluoro-N-[2-(2-fluoro-4-{[4-(trifluoromethyl)pyridin-2-yl]oxy}phenyl)ethyl]quinazolin-4-amine (1210070-84-0) (WO2010025451), (2.29) N-[9-(dichloromethylene)-1,2,3,4-tetrahydro-1,4-methanonaphthalen-5-yl]-3-(difluoromethyl)-1-methyl-1H-pyrazole-4-carboxamide, (2.30) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and (2.31) N—[(1R,4S)-9-(dichloromethylene)-1,2,3,4-tetrahydro-1,4-methanonaphthalen-5-yl]-3-(difluoromethyl)-1-methyl-1H-pyrazole-4-carboxamide.
(3) inhibitors of the respiratory chain at complex III, for example (3.1) ametoctradin (865318-97-4), (3.2) amisulbrom (348635-87-0), (3.3) azoxystrobin (131860-33-8), (3.4) cyazofamid (120116-88-3), (3.5) coumethoxystrobin (850881-30-0), (3.6) coumoxystrobin (850881-70-8), (3.7) dimoxystrobin (141600-52-4), (3.8) enestroburin (238410-11-2) (WO 2004/058723), (3.9) famoxadone (131807-57-3) (WO 2004/058723), (3.10) fenamidone (161326-34-7) (WO 2004/058723), (3.11) fenoxystrobin (918162-02-4), (3.12) fluoxastrobin (361377-29-9) (WO 2004/058723), (3.13) kresoxim-methyl (143390-89-0) (WO 2004/058723), (3.14) metominostrobin (133408-50-1) (WO 2004/058723), (3.15) orysastrobin (189892-69-1) (WO 2004/058723), (3.16) picoxystrobin (117428-22-5) (WO 2004/058723), (3.17) pyraclostrobin (175013-18-0) (WO 2004/058723), (3.18) pyrametostrobin (915410-70-7) (WO 2004/058723), (3.19) pyraoxystrobin (862588-11-2) (WO 2004/058723), (3.20) pyribencarb (799247-52-2) (WO 2004/058723), (3.21) triclopyricarb (902760-40-1), (3.22) trifloxystrobin (141517-21-7) (WO 2004/058723), (3.23) (2E)-2-(2-{[6-(3-chloro-2-methylphenoxy)-5-fluoropyrimidin-4-yl]oxy}phenyl)-2-(methoxyimino)-N-methylethanamide (WO 2004/058723), (3.24) (2E)-2-(methoxyimino)-N-methyl-2-(2-{[({(1E)-1-[3-(trifluoromethyl)phenyl]ethylidene}amino)oxy]methyl}phenyl)ethanamide (WO 2004/058723), (3.25) (2E)-2-(methoxyimino)-N-methyl-2-{2-[(E)-({1-[3-(trifluoromethyl)phenyl]ethoxy}imino)methyl]phenyl}ethanamide (158169-73-4), (3.26) (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide (326896-28-0), (3.27) (2E)-2-{2-[({[(2E,3E)-4-(2,6-dichlorophenyl)but-3-en-2-ylidene]amino}oxy)methyl]phenyl}-2-(methoxyimino)-N-methylethanamide, (3.28) 2-chloro-N-(1,1,3-trimethyl-2,3-dihydro-1H-inden-4-yl)pyridine-3-carboxamide (119899-14-8), (3.29) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (3.30) methyl (2E)-2-{2-[({cyclopropyl[(4-methoxyphenyl)imino]methyl}sulfanyl)methyl]phenyl}-3-methoxyprop-2-enoate (149601-03-6), (3.31) N-(3-ethyl-3,5,5-trimethylcyclohexyl)-3-(formylamino)-2-hydroxybenzamide (226551-21-9), (3.32) 2-{2-[(2,5-dimethylphenoxy)methyl]phenyl}-2-methoxy-N-methylacetamide (173662-97-0) and (3.33) (2R)-2-{2-[(2,5-dimethylphenoxy)methyl]phenyl}-2-methoxy-N-methylacetamide (394657-24-0).
(4) Inhibitors of the mitosis and cell division, for example (4.1) benomyl (17804-35-2), (4.2) carbendazim (10605-21-7), (4.3) chlorfenazole (3574-96-7), (4.4) diethofencarb (87130-20-9), (4.5) ethaboxam (162650-77-3), (4.6) fluopicolide (239110-15-7), (4.7) fuberidazole (3878-19-1), (4.8) pencycuron (66063-05-6), (4.9) thiabendazole (148-79-8), (4.10) thiophanate-methyl (23564-05-8), (4.11) thiophanate (23564-06-9), (4.12) zoxamide (156052-68-5), (4.13) 5-chloro-7-(4-methylpiperidin-1-yl)-6-(2,4,6-trifluorophenyl)[1,2,4]triazolo[1,5-a]pyrimidine (214706-53-3) and (4.14) 3-chloro-5-(6-chloropyridin-3-yl)-6-methyl-4-(2,4,6-trifluorophenyl)pyridazine (1002756-87-7).
(5) Compounds capable to have a multisite action, like for example (5.1) bordeaux mixture (8011-63-0),

(5.2) captafol (2425-06-1), (5.3) captan (133-06-2) (WO 02/12172), (5.4) chlorothalonil (1897-45-6), (5.5) copper hydroxide (20427-59-2), (5.6) copper naphthenate (1338-02-9), (5.7) copper oxide (1317-39-1), (5.8) copper oxychloride (1332-40-7), (5.9) copper(2+) sulfate (7758-98-7), (5.10) dichlofluanid (1085-98-9), (5.11) dithianon (3347-22-6), (5.12) dodine (2439-10-3), (5.13) dodine free base, (5.14) ferbam (14484-64-1), (5.15) fluorofolpet (719-96-0), (5.16) folpet (133-07-3), (5.17) guazatine (108173-90-6), (5.18) guazatine acetate, (5.19) iminoctadine (13516-27-3), (5.20) iminoctadine albesilate (169202-06-6), (5.21) iminoctadine triacetate (57520-17-9), (5.22) mancopper (53988-93-5), (5.23) mancozeb (8018-01-7), (5.24) maneb (12427-38-2), (5.25) metiram (9006-42-2), (5.26) metiram zinc (9006-42-2), (5.27) oxine-copper (10380-28-6), (5.28) propamidine (104-32-5), (5.29) propineb (12071-83-9), (5.30) sulphur and sulphur preparations including calcium polysulphide (7704-34-9), (5.31) thiram (137-26-8), (5.32) tolylfluanid (731-27-1), (5.33) zineb (12122-67-7) and (5.34) ziram (137-30-4).

(6) Compounds capable to induce a host defence, for example (6.1) acibenzolar-5-methyl (135158-54-2), (6.2) isotianil (224049-04-1), (6.3) probenazole (27605-76-1) and (6.4) tiadinil (223580-51-6).
(7) Inhibitors of the amino acid and/or protein biosynthesis, for example (7.1) andoprim (23951-85-1), (7.2) blasticidin-S (2079-00-7), (7.3) cyprodinil (121552-61-2), (7.4) kasugamycin (6980-18-3), (7.5) kasugamycin hydrochloride hydrate (19408-46-9), (7.6) mepanipyrim (110235-47-7), (7.7) pyrimethanil (53112-28-0) and (7.8) 3-(5-fluoro-3,3,4,4-tetramethyl-3,4-dihydroisoquinolin-1-yl)quinoline (861647-32-7) (WO2005070917).
(8) Inhibitors of the ATP production, for example (8.1) fentin acetate (900-95-8), (8.2) fentin chloride (639-58-7), (8.3) fentin hydroxide (76-87-9) and (8.4) silthiofam (175217-20-6).
(9) Inhibitors of the cell wall synthesis, for example (9.1) benthiavalicarb (177406-68-7), (9.2) dimethomorph (110488-70-5), (9.3) flumorph (211867-47-9), (9.4) iprovalicarb (140923-17-7), (9.5) mandipropamid (374726-62-2), (9.6) polyoxins (11113-80-7), (9.7) polyoxorim (22976-86-9), (9.8) validamycin A (37248-47-8) and (9.9) valifenalate (283159-94-4; 283159-90-0).
(10) Inhibitors of the lipid and membrane synthesis, for example (10.1) biphenyl (92-52-4), (10.2) chloroneb (2675-77-6), (10.3) dicloran (99-30-9), (10.4) edifenphos (17109-49-8), (10.5) etridiazole (2593-15-9), (10.6) iodocarb (55406-53-6), (10.7) iprobenfos (26087-47-8), (10.8) isoprothiolane (50512-35-1), (10.9) propamocarb (25606-41-1), (10.10) propamocarb hydrochloride (25606-41-1), (10.11) prothiocarb (19622-08-3), (10.12) pyrazophos (13457-18-6), (10.13) quintozene (82-68-8), (10.14) tecnazene (117-18-0) and (10.15) tolclofos-methyl (57018-04-9).
(11) Inhibitors of the melanine biosynthesis, for example (11.1) carpropamid (104030-54-8), (11.2) diclocymet (139920-32-4), (11.3) fenoxanil (115852-48-7), (11.4) phthalide (27355-22-2), (11.5) pyroquilon (57369-32-1), (11.6) tricyclazole (41814-78-2) and (11.7) 2,2,2-trifluoroethyl {3-methyl-1-[(4-methylbenzoyl)amino]butan-2-yl}carbamate (851524-22-6) (WO2005042474).
(12) Inhibitors of the nucleic acid synthesis, for example (12.1) benalaxyl (71626-11-4), (12.2) benalaxyl-M (kiralaxyl) (98243-83-5), (12.3) bupirimate (41483-43-6), (12.4) clozylacon (67932-85-8), (12.5) dimethirimol (5221-53-4), (12.6) ethirimol (23947-60-6), (12.7) furalaxyl (57646-30-7), (12.8) hymexazol (10004-44-1), (12.9) metalaxyl (57837-19-1), (12.10) metalaxyl-M (mefenoxam) (70630-17-0), (12.11) ofurace (58810-48-3), (12.12) oxadixyl (77732-09-3) and (12.13) oxolinic acid (14698-29-4).
(13) Inhibitors of the signal transduction, for example (13.1) chlozolinate (84332-86-5), (13.2) fenpiclonil (74738-17-3), (13.3) fludioxonil (131341-86-1), (13.4) iprodione (36734-19-7), (13.5) procymidone (32809-16-8), (13.6) quinoxyfen (124495-18-7) and (13.7) vinclozolin (50471-44-8).
(14) Compounds capable to act as an uncoupler, for example (14.1) binapacryl (485-31-4), (14.2) dinocap (131-72-6), (14.3) ferimzone (89269-64-7), (14.4) fluazinam (79622-59-6) and (14.5) meptyldinocap (131-72-6).
(15) Further compounds, for example (15.1) benthiazole (21564-17-0), (15.2) bethoxazin (163269-30-5), (15.3) capsimycin (70694-08-5), (15.4) carvone (99-49-0), (15.5) chinomethionat (2439-01-2), (15.6) pyriofenone (chlazafenone) (688046-61-9), (15.7) cufraneb (11096-18-7), (15.8) cyflufenamid (180409-60-3), (15.9) cymoxanil (57966-95-7), (15.10) cyprosulfamide (221667-31-8), (15.11) dazomet (533-74-4), (15.12) debacarb (62732-91-6), (15.13) dichlorophen (97-23-4), (15.14) diclomezine (62865-36-5), (15.15) difenzoquat (49866-87-7), (15.16) difenzoquat methylsulphate (43222-48-6), (15.17) diphenylamine (122-39-4), (15.18) ecomate, (15.19) fenpyrazamine (473798-59-3), (15.20) flumetover (154025-04-4), (15.21) fluoroimide (41205-21-4), (15.22) flusulfamide (106917-52-6), (15.23) flutianil (304900-25-2), (15.24) fosetyl-aluminium (39148-24-8), (15.25) fosetyl-calcium, (15.26) fosetyl-sodium (39148-16-8), (15.27) hexachlorobenzene (118-74-1), (15.28) irumamycin (81604-73-1), (15.29) methasulfocarb (66952-49-6), (15.30) methyl isothiocyanate (556-61-6), (15.31) metrafenone (220899-03-6), (15.32) mildiomycin (67527-71-3), (15.33) natamycin (7681-93-8), (15.34) nickel dimethyldithiocarbamate (15521-65-0), (15.35) nitrothal-isopropyl (10552-74-6), (15.36) octhilinone (26530-20-1), (15.37) oxamocarb (917242-12-7), (15.38) oxyfenthiin (34407-87-9), (15.39) pentachlorophenol and salts (87-86-5), (15.40) phenothrin, (15.41) phosphorous acid and its salts (13598-36-2), (15.42) propamocarb-fosetylate, (15.43) propanosine-sodium (88498-02-6), (15.44) proquinazid (189278-12-4), (15.45) pyrimorph (868390-90-3), (15.45e) (2E)-3-(4-tert-butylphenyl)-3-(2-chloropyridin-4-yl)-1-(morpholin-4-yl)prop-2-en-1-one (1231776-28-5), (15.45z) (2Z)-3-(4-tert-butylphenyl)-3-(2-chloropyridin-4-yl)-1-(morpholin-4-yl)prop-2-en-1-one (1231776-29-6), (15.46) pyrrolnitrine (1018-71-9) (EP-A 1 559 320), (15.47) tebufloquin (376645-78-2), (15.48) tecloftalam (76280-91-6), (15.49) tolnifanide (304911-98-6), (15.50) triazoxide (72459-58-6), (15.51) trichlamide (70193-21-4), (15.52) zarilamid (84527-51-5), (15.53) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate (517875-34-2) (WO2003035617), (15.54) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone (1003319-79-6) (WO 2008013622), (15.55) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone (1003319-80-9) (WO 2008013622), (15.56) 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone (1003318-67-9) (WO 2008013622), (15.57) 1-(4-methoxyphenoxy)-3,3-dimethylbutan-2-yl 1H-imidazole-1-carboxylate (111227-17-9), (15.58) 2,3,5,6-tetrachloro-4-(methylsulfonyl)pyridine (13108-52-6), (15.59) 2,3-dibutyl-6-chlorothieno[2,3-d]pyrimidin-4(3H)-one (221451-58-7), (15.60) 2,6-dimethyl-1H,5H-[1,4]dithiino[2,3-c:5,6-c′]dipyrrole-1,3,5,7(2H,6H)-tetrone, (15.61) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone (1003316-53-7) (WO 2008013622), (15.62) 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone (1003316-54-8) (WO 2008013622), (15.63) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone (1003316-51-5) (WO 2008013622), (15.64) 2-butoxy-6-iodo-3-propyl-4H-chromen-4-one, (15.65) 2-chloro-5-[2-chloro-1-(2,6-difluoro-4-methoxyphenyl)-4-methyl-1H-imidazol-5-yl]pyridine, (15.66) 2-phenylphenol and salts (90-43-7), (15.67) 3-(4,4,5-trifluoro-3,3-dimethyl-3,4-dihydroisoquinolin-1-yl)quinoline (861647-85-0) (WO2005070917), (15.68) 3,4,5-trichloropyridine-2,6-dicarbonitrile (17824-85-0), (15.69) 3-[5-(4-chlorophenyl)-2,3-dimethyl-1,2-oxazolidin-3-yl]pyridine, (15.70) 3-chloro-5-(4-chlorophenyl)-4-(2,6-difluorophenyl)-6-methylpyridazine, (15.71) 4-(4-chlorophenyl)-5-(2,6-difluorophenyl)-3,6-dimethylpyridazine, (15.72) 5-amino-1,3,4-thiadiazole-2-thiol, (15.73) 5-chloro-N′-phenyl-N′-(prop-2-yn-1-yl)thiophene-2-sulfonohydrazide (134-31-6), (15.74) 5-fluoro-2-[(4-fluorobenzyl)oxy]pyrimidin-4-amine (1174376-11-4) (WO2009094442), (15.75) 5-fluoro-2-[(4-methylbenzyl)oxy]pyrimidin-4-amine (1174376-25-0) (WO2009094442), (15.76) 5-methyl-6-octyl[1,2,4]triazolo[1,5-a]pyrimidin-7-amine, (15.77) ethyl (2Z)-3-amino-2-cyano-3-phenylprop-2-enoate, (15.78) N′-(4-{[3-(4-chlorobenzyl)-1,2,4-thiadiazol-5-yl]oxy)}-2,5-dimethylphenyl)-N-ethyl-N-methylimidoformamide, (15.79) N-(4-chlorobenzyl)-3-[3-methoxy-4-(prop-2-yn-1-yloxy)phenyl]propanamide, (15.80) N-[(4-chlorophenyl)(cyano)methyl]-3-[3-methoxy-4-(prop-2-yn-1-yloxy)phenyl]propanamide, (15.81) N-[(5-bromo-3-chloropyridin-2-yl)methyl]-2,4-dichloropyridine-3-carboxamide, (15.82) N-[1-(5-bromo-3-chloropyridin-2-yl)ethyl]-2,4-dichloropyridine-3-carboxamide, (15.83) N-[1-(5-bromo-3-chloropyridin-2-yl)ethyl]-2-fluoro-4-iodopyridine-3-carboxamide, (15.84) N-{(E)-[(cyclopropylmethoxy)imino][6-(difluoromethoxy)-2,3-difluorophenyl]methyl}-2-phenylacetamide (221201-92-9), (15.85) N-{(Z)-[(cyclopropylmethoxy)imino][6-(difluoromethoxy)-2,3-difluorophenyl]methyl})-2-phenylacetamide (221201-92-9), (15.86) N′-{4-[(3-tert-butyl-4-cyano-1,2-thiazol-5-yl)oxy]-2-chloro-5-methylphenyl}-N-ethyl-N-methylimidoformamide, (15.87) N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide (922514-49-6) (WO 2007014290), (15.88) N-methyl-2-(1-{[5-methyl-3-(trifluoromethyl)-1H-pyrazol-1-yl]acetyl}piperidin-4-yl)-N—[(1R)-1,2,3,4-tetrahydronaphthalen-1-yl]-1,3-thiazole-4-carboxamide (922514-07-6) (WO 2007014290), (15.89) N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide (922514-48-5) (WO 2007014290), (15.90) pentyl {6-[({[(1-methyl-1H-tetrazol-5-yl)(phenyl)methylidene]amino}oxy)methyl]pyridin-2-yl}carbamate, (15.91) phenazine-1-carboxylic acid, (15.92) quinolin-8-ol (134-31-6), (15.93) quinolin-8-ol sulfate (2:1) (134-31-6) and (15.94) tert-butyl {6-[({[(1-methyl-1H-tetrazol-5-yl)(phenyl)methylene]amino}oxy)methyl]pyridin-2-yl}carbamate.
(16) Further compounds, for example (16.1) 1-methyl-3-(trifluoromethyl)-N-[2′-(trifluoromethyl)biphenyl-2-yl]-1H-pyrazole-4-carboxamide, (16.2) N-(4′-chlorobiphenyl-2-yl)-3-(difluoromethyl)-1-methyl-1H-pyrazole-4-carboxamide, (16.3) N-(2′,4′-dichlorobiphenyl-2-yl)-3-(difluoromethyl)-1-methyl-1H-pyrazole-4-carboxamide, (16.4) 3-(difluoromethyl)-1-methyl-N-[4′-(trifluoromethyl)biphenyl-2-yl]-1H-pyrazole-4-carboxamide, (16.5) N-(2′,5′-difluorobiphenyl-2-yl)-1-methyl-3-(trifluoromethyl)-1H-pyrazole-4-carboxamide, (16.6) 3-(difluoromethyl)-1-methyl-N-[4′-(prop-1-yn-1-yl)biphenyl-2-yl]-1H-pyrazole-4-carboxamide (known from WO 2004/058723), (16.7) 5-fluoro-1,3-dimethyl-N-[4′-(prop-1-yn-1-yl)biphenyl-2-yl]-1H-pyrazole-4-carboxamide (known from WO 2004/058723), (16.8) 2-chloro-N-[4′-(prop-1-yn-1-yl)biphenyl-2-yl]pyridine-3-carboxamide (known from WO 2004/058723), (16.9) 3-(difluoromethyl)-N-[4′-(3,3-dimethylbut-1-yn-1-yl)biphenyl-2-yl]-1-methyl-1H-pyrazole-4-carboxamide (known from WO 2004/058723), (16.10) N-[4′-(3,3-dimethylbut-1-yn-1-yl)biphenyl-2-yl]-5-fluoro-1,3-dimethyl-1H-pyrazole-4-carboxamide (known from WO 2004/058723), (16.11) 3-(difluoromethyl)-N-(4′-ethynylbiphenyl-2-yl)-1-methyl-1H-pyrazole-4-carboxamide (known from WO 2004/058723), (16.12) N-(4′-ethynylbiphenyl-2-yl)-5-fluoro-1,3-dimethyl-1H-pyrazole-4-carboxamide (known from WO 2004/058723), (16.13) 2-chloro-N-(4′-ethynylbiphenyl-2-yl)pyridine-3-carboxamide (known from WO 2004/058723), (16.14) 2-chloro-N-[4′-(3,3-dimethylbut-1-yn-1-yl)biphenyl-2-yl]pyridine-3-carboxamide (known from WO 2004/058723), (16.15) 4-(difluoromethyl)-2-methyl-N-[4′-(trifluoromethyl)biphenyl-2-yl]-1,3-thiazole-5-carboxamide (known from WO 2004/058723), (16.16) 5-fluoro-N-[4′-(3-hydroxy-3-methylbut-1-yn-1-yl)biphenyl-2-yl]-1,3-dimethyl-1H-pyrazole-4-carboxamide (known from WO 2004/058723), (16.17) 2-chloro-N-[4′-(3-hydroxy-3-methylbut-1-yn-1-yl)biphenyl-2-yl]pyridine-3-carboxamide (known from WO 2004/058723), (16.18) 3-(difluoromethyl)-N-[4′-(3-methoxy-3-methylbut-1-yn-1-yl)biphenyl-2-yl]-1-methyl-1H-pyrazole-4-carboxamide (known from WO 2004/058723), (16.19) 5-fluoro-N-[4′-(3-methoxy-3-methylbut-11-yn-1-yl)biphenyl-2-yl]-1,3-dimethyl-1H-pyrazole-4-carboxamide (known from WO 2004/058723), (16.20) 2-chloro-N-[4′-(3-methoxy-3-methylbut-1-yn-1-yl)biphenyl-2-yl]pyridine-3-carboxamide (known from WO 2004/058723), (16.21) (5-bromo-2-methoxy-4-methylpyridin-3-yl)(2,3,4-trimethoxy-6-methylphenyl)methanone (known from EP-A 1 559 320), (16.22) N-[2-(4-{[3-(4-chlorophenyl)prop-2-yn-1-yl]oxy}-3-methoxyphenyl)ethyl]-N2-(methylsulfonyl)valinamide (220706-93-4), (16.23) 4-oxo-4-[(2-phenylethyl)amino]butanoic acid and (16.24) but-3-yn-1-yl {6-[({[(Z)-(1-methyl-1H-tetrazol-5-yl)(phenyl)methylene]amino}oxy)methyl]pyridin-2-yl}carbamate.

All named mixing partners of the classes (1) to (16) can, if their functional groups enable this, optionally form salts with suitable bases or acids.

According to the invention all plants and plant material can be treated. By plants is meant all plants and plant populations such as desirable and undesirable wild plants, cultivars (including naturally occurring cultivars) and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random and directed mutagenesis, molecular or genetic markers or by bioengineering and genetic engineering methods including transgenic plants.

By plant material is meant all above ground and below ground parts and organs of plants such as shoot, leaf, flower, blossom and root, whereby for example leaves, needles, stems, branches, blossoms, fruiting bodies, fruits and seed as well as roots, corms and rhizomes are listed.

In a particular embodiment the plant material to be treated are leaves, shoots, flowers, grains, seeds.

In a particular embodiment the plant material to be treated are leaves, shoots, flowers, grains, seeds.

By ‘plant propagation material’ is meant generative and vegetative parts of a plant including seeds of all kinds (fruit, tubers, bulbs, grains etc), runners, pods, fruiting bodies, roots, rhizomes, cuttings, corms, cut shoots and the like.

Plant propagation material may also include plants and young plants which are to be transplanted after germination or after emergence from the soil.

Among the plants that can be protected by the method according to the invention, mention may be made of major field crops like corn, soybean, cotton, Brassica oilseeds such as Brassica napus (e.g. canola), Brassica rapa, B. juncea (e.g. mustard) and Brassica carinata, rice, wheat, sugarbeet, sugarcane, oats, rye, barley, millet, triticale, flax, vine and various fruits and vegetables of various botanical taxa such as Rosaceae sp. (for instance pip fruit such as apples and pears, but also stone fruit such as apricots, cherries, almonds and peaches, berry fruits such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for instance banana trees and plantings), Rubiaceae sp. (for instance coffee), Theaceae sp., Sterculiceae sp., Rutaceae sp. (for instance lemons, oranges and grapefruit); Solanaceae sp. (for instance tomatoes, potatoes, peppers, eggplant), Liliaceae sp., Compositiae sp. (for instance lettuce, artichoke and chicory—including mot chicory, endive or common chicory), Umbelliferae sp. (for instance carrot, parsley, celery and celeriac), Cucurbitaceae sp. (for instance cucumber—including pickling cucumber, squash, watermelon, gourds and melons), Alliaceae sp. (for instance onions and leek), Cruciferae sp. (for instance white cabbage, red cabbage, broccoli, cauliflower, brussel sprouts, pak choi, kohlrabi, radish, horseradish, cress, Chinese cabbage), Leguminosae sp. (for instance peanuts, peas and beans beans—such as climbing beans and broad beans), Chenopodiaceae sp. (for instance mangold, spinach beet, spinach, beetroots), Malvaceae (for instance okra), Asparagaceae (for instance asparagus); horticultural and forest crops; ornamental plants; as well as genetically modified homologues of these crops.

In a particular embodiment crops from the family of Poaceae which is comprised of wheat, oat, barley, rye, triticale, millet, corn, maize can be protected by the method of the invention.

The methods, compounds and compositions of the present invention are suitable for reducing mycotoxin contamination on a number of plants and their propagation material including, but not limited to the following target crops: vine, flaxcotton, cereals (wheat, barley, rye, oats, millet, triticale, maize (including field corn, pop corn and sweet corn), rice, sorghum and related crops); beet (sugar beet and fodder beet); sugar beet, sugar cane, leguminous plants (beans, lentils, peas, soybeans); oil plants (rape, mustard, sunflowers), Brassica oilseeds such as Brassica napus (e.g. canola), Brassica rapa, B. juncea (e.g. mustard) and Brassica carinata; cucumber plants (marrows, cucumbers, melons); fibre plants (cotton, flax, hemp, jute); vegetables (spinach, lettuce, asparagus, cabbages, carrots, eggplants, onions, pepper, tomatoes, potatoes, paprika, okra); plantation crops (bananas, fruit trees, rubber trees, tree nurseries), ornamentals (flowers, shrubs, broad-leaved trees and evergreens, such as conifers); as well as other plants such as vines, bushberries (such as blueberries), caneberries, cranberries, peppermint, rhubarb, spearmint, sugar cane and turf grasses including, but not limited to, cool-season turf grasses (for example, bluegrasses (Poa L.), such as Kentucky bluegrass (Poa pratensis L.), rough bluegrass (Poa trivialis L.), Canada bluegrass (Poa compressa L.) and annual bluegrass (Poa annua L.); bentgrasses (Agrostis L.), such as creeping bentgrass (Agrostis palustris Huds.), colonial bentgrass (Agrostis tenius Sibth.), velvet bentgrass (Agrostis canina L.) and redtop (Agrostis alba L.); fescues (Festuca L.), such as tall fescue (Festuca arundinacea Schreb.), meadow fescue (Festuca elatior L.) and fine fescues such as creeping red fescue (Festuca rubra L.), chewings fescue (Festuca rubra var. commutata Gaud.), sheep fescue (Festuca ovina L.) and hard fescue (Festuca longifolia); and ryegrasses (Lolium L.), such as perennial ryegrass (Lolium perenne L.) and annual (Italian) ryegrass (Lolium multiflorum Lam.)) and warm-season turf grasses (for example, Bermudagrasses (Cynodon L. C. Rich), including hybrid and common Bermudagrass; Zoysiagrasses (Zoysia Willd.), St. Augustinegrass (Stenotaphrum secundatum (Walt.) Kuntze); and centipedegrass (Eremochloa ophiuroides (Munro.) Hack.)); various fruits and vegetables of various botanical taxa such as Rosaceae sp. (for instance pip fruit such as apples and pears, but also stone fruit such as apricots, cherries, almonds and peaches, berry fruits such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for instance banana trees and plantings), Rubiaceae sp. (for instance coffee), Theaceae sp., Sterculiceae sp, Rutaceae sp. (for instance lemons, oranges and grapefruit); Solanaceae sp. (for instance tomatoes, potatoes, peppers, eggplant), Liliaceae sp., Compositiae sp. (for instance lettuce, artichoke and chicory—including root chicory, endive or common chicory), Umbelliferae sp. (for instance carrot, parsley, celery and celeriac), Cucurbitaceae sp. (for instance cucumber—including pickling cucumber, squash, watermelon, gourds and melons), Alliaceae sp. (for instance onions and leek), Cruciferae sp. (for instance white cabbage, red cabbage, broccoli, cauliflower, brussel sprouts, pak choi, kohlrabi, radish, horseradish, cress, Chinese cabbage), Leguminosae sp. (for instance peanuts, peas and beans beans—such as climbing beans and broad beans), Chenopodiaceae sp. (for instance mangold, spinach beet, spinach, beetroots), Malvaceae (for instance okra), Asparagaceae (for instance asparagus); horticultural and forest crops; ornamental plants; as well as genetically modified homologues of these crops.

The method of treatment according to the invention can be used in the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, co suppression technology or RNA interference—RNAi—technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates, the active compound combinations according to the invention may also have a strengthening effect in plants. Accordingly, they are also suitable for mobilizing the defense system of the plant against attack by unwanted phytopathogenic fungi and/or microorganisms and/or viruses. This may, if appropriate, be one of the reasons of the enhanced activity of the combinations according to the invention, for example against fungi. Plant-strengthening (resistance-inducing) substances are to be understood as meaning, in the present context, those substances or combinations of substances which are capable of stimulating the defense system of plants in such a way that, when subsequently inoculated with unwanted phytopathogenic fungi and/or microorganisms and/or viruses, the treated plants display a substantial degree of resistance to these unwanted phytopathogenic fungi and/or microorganisms and/or viruses. In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozon exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stress factors. Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling, i.e. the mechanical removal of the male reproductive organs (or males flowers) but, more typically, male sterility is the result of genetic determinants in the plant genome. In that case, and especially when seed is the desired product to be harvested from the hybrid plants it is typically useful to ensure that male fertility in the hybrid plants is fully restored. This can be accomplished by ensuring that the male parents have appropriate fertility restorer genes which are capable of restoring the male fertility in hybrid plants that contain the genetic determinants responsible for male-sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male-sterile plants is described in WO 1989/10396 in which, for example, a ribonuclease such as barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance. Herbicide-tolerant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a Petunia EPSPS, a Tomato EPSPS, or an Eleusine EPSPS (WO 2001/66704). It can also be a mutated EPSPS. Glyphosate-tolerant plants can also be obtained by expressing a gene that encodes a glyphosate oxido-reductase enzyme. Glyphosate-tolerant plants can also be obtained by expressing a gene that encodes a glyphosate acetyl transferase enzyme. Glyphosate-tolerant plants can also be obtained by selecting plants containing naturally-occurring mutations of the above-mentioned genes.

Other herbicide resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is an enzyme encoding a phosphinothricin acetyltransferase (such as the bar or pat protein from Streptomyces species). Plants expressing an exogenous phosphinothricin acetyltransferase are described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Still further herbicide resistant plants are plants that are made tolerant to acetolactate synthase (ALS) inhibitors. Known ALS-inhibitors include, for example, sulfonylurea, imidazolinone, triazolopyrimidines, pyrimidinyloxy(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides. The production of sulfonylurea-tolerant plants and imidazolinone-tolerant plants is described. Other imidazolinone-tolerant plants are also described. Further sulfonylurea- and imidazolinone-tolerant plants are also described.

Other plants tolerant to imidazolinone and/or sulfonylurea can be obtained by induced mutagenesis, selection in cell cultures in the presence of the herbicide or mutation breeding as described for soybeans, for rice, for sugar beet, for lettuce, or for sunflower.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

An “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1F, Cry2Ab, Cry3Aa, or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins; or
3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON98034; or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bbl protein in corn events MON863 or MON88017, or the Cry3A protein in corn event MIR604;
5) an insecticidal secreted protein from Bacillus thuringiensis or Bacillus cereus, or an insecticidal portion thereof, such as the vegetative insecticidal (VIP) proteins listed at:
   http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins; or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102.

Of course, an insect-resistant transgenic plant, as used herein, also includes any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected when using different proteins directed at different target insect species, or to delay insect resistance development to the plants by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of poly(ADP-ribose)polymerase (PARP) gene in the plant cells or plants.
b. plants which contain a stress tolerance enhancing transgene capable of reducing the expression and/or the activity of the PARG encoding genes of the plants or plants cells.
c. plants which contain a stress tolerance enhancing transgene coding for a plant-functional enzyme of the nicotinamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphoribosyltransferase.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesised starch in wild type plant cells or plants, so that this is better suited for special applications. Said transgenic plants synthesizing a modified starch are disclosed.
2) transgenic plants which synthesize non starch carbohydrate polymers or which synthesize non starch carbohydrate polymers with altered properties in comparison to wild type plants without genetic modification. Examples are plants producing polyfructose, especially of the inulin and levan-type, plants producing alpha 1,4 glucans, plants producing alpha-1,6 branched alpha-1,4-glucans, plants producing alternan,
3) transgenic plants which produce hyaluronan.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics and include:
a) Plants, such as cotton plants, containing an altered form of cellulose synthase genes,
b) Plants, such as cotton plants, containing an altered form of rsw2 or rsw3 homologous nucleic acids,
c) Plants, such as cotton plants, with increased expression of sucrose phosphate synthase,
d) Plants, such as cotton plants, with increased expression of sucrose synthase,

e) Plants, such as cotton plants, wherein the timing of the plasmodesmatal gating at the basis of the fiber cell is altered, e.g. through downregulation of fiber selective β 1,3-glucanase,
f) Plants, such as cotton plants, having fibers with altered reactivity, e.g. through the expression of N-acteylglucosaminetransferase gene including nodC and chitinsynthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants contain a mutation imparting such altered oil characteristics and include:
a) Plants, such as oilseed rape plants, producing oil having a high oleic acid content,
b) Plants such as oilseed rape plants, producing oil having a low linolenic acid content,
c) Plant such as oilseed rape plants, producing oil having a low level of saturated fatty acids.

Particularly useful transgenic plants which may be treated according to the invention are plants which comprise one or more genes which encode one or more toxins, such as the following which are sold under the trade names YIELD GARD® (for example maize, cotton, soya beans), KnockOut® (for example maize), BiteGard® (for example maize), Bt-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or combination of transformation events, that are listed for example in the databases from various national or regional regulatory agencies (see for example http://gmoinfo.jrc.it/gmp_browse.aspx and http://www.agbios.com/dbase.php).

TABLE A

| | Transgenic | | | |
|---|---|---|---|---|
| No. | event | Company | Description | Crop |
| A-1 | ASR368 | Scotts Seeds | Glyphosate tolerance derived by inserting a modified 5-enolpyruvylshikimate- | Agrostis stolonifera |
| | | | 3-phosphate synthase (EPSPS) encoding gene from Agrobacterium | Creeping Bentgrass |
| | | | tumefaciens. | |
| A-2 | H7-1 | Monsanto Company | Glyphosate herbicide tolerant sugar beet produced by inserting a gene | Beta vulgaris |
| | | | encoding the enzyme 5-enolypyruvylshikimate-3-phosphate synthase (EPSPS) | |
| | | | from the CP4 strain of Agrobacterium tumefaciens. | |
| A-3 | T120-7 | Bayer CropScience | Introduction of the PPT-acetyltransferase (PAT) encoding gene from | Beta vulgaris |
| | | (Aventis | Streptomyces viridochromogenes, an aerobic soil bacteria. PPT normally acts | |
| | | CropScience(AgrEvo)) | to inhibit glutamine synthetase, causing a fatal accumulation of ammonia. | |
| | | | Acetylated PPT is inactive. | |
| A-4 | GTSB77 | Novartis Seeds; | Glyphosate herbicide tolerant sugar beet produced by inserting a gene | Beta vulgaris sugar |
| | | Monsanto Company | encoding the enzyme 5-enolypyruvylshikimate-3-phosphate synthase (EPSPS) | Beet |
| | | | from the CP4 strain of Agrobacterium tumefaciens. | |
| A-5 | 23-18-17, | Monsanto Company | High laurate (12:0) and myristate (14:0) canola produced by inserting a | Brassica |
| | 23-198 | (formerly Calgene) | thioesterase encoding gene from the California bay laurel (Umbellularia | napus (Argentine |
| | | | californica). | Canola) |
| A-6 | 45A37, | Pioneer Hi-Bred | High oleic acid and low linolenic acid canola produced through a combination | Brassica |
| | 46A40 | International Inc. | of chemical mutagenesis to select for a fatty acid desaturase mutant with | napus (Argentine |
| | | | elevated oleic acid, and traditional back-crossing to introduce the low linolenic | Canola) |
| | | | acid trait. | |
| A-7 | 46A12, | Pioneer Hi-Bred | Combination of chemical mutagenesis, to achieve the high oleic acid trait, and | Brassica |
| | 46A16 | International Inc. | traditional breeding with registered canola varieties. | napus (Argentine |
| | | | | Canola) |
| A-8 | GT200 | Monsanto Company | Glyphosate herbicide tolerant canola produced by inserting genes encoding the | Brassica |
| | | | enzymes 5-enolypyruvylshikimate-3-phosphate synthase (EPSPS) from the | napus (Argentine |
| | | | CP4 strain of Agrobacterium tumefaciens and glyphosate oxidase from | Canola) |
| | | | Ochrobactrum anthropi. | |
| A-9 | GT73, | Monsanto Company | Glyphosate herbicide tolerant canola produced by inserting genes encoding the | Brassica |
| | RT73 | | enzymes 5-enolypyruvylshikimate-3-phosphate synthase (EPSPS) from the | napus (Argentine |
| | | | CP4 strain of Agrobacterium tumefaciens and glyphosate oxidase from | Canola) |
| | | | Ochrobactrum anthropi. | |
| A-10 | HCN10 | Aventis CropScience | Introduction of the PPT-acetyltransferase (PAT) encoding gene from | Brassica |
| | | | Streptomyces viridochromogenes, an aerobic soil bacteria. PPT normally acts | napus (Argentine |
| | | | to inhibit glutamine synthetase, causing a fatal accumulation of ammonia. | Canola) |
| | | | Acetylated PPT is inactive. | |
| A-11 | HCN92 | Bayer CropScience | Introduction of the PPT-acetyltransferase (PAT) encoding gene from | Brassica |
| | | (Aventis | Streptomyces viridochromogenes, an aerobic soil bacteria. PPT normally acts | napus (Argentine |
| | | CropScience(AgrEvo)) | to inhibit glutamine synthetase, causing a fatal accumulation of ammonia. | Canola) |
| | | | Acetylated PPT is inactive. | |
| A-12 | MS1, RF1 | Aventis CropScience | Male-sterility, fertility restoration, pollination control system displaying | Brassica |
| | =>PGS1 | (formerly Plant | glufosinate herbicide tolerance. MS lines contained the barnase gene from | napus (Argentine |
| | | Genetic Systems) | Bacillus amyloliquefaciens, RF lines contained the barstar gene from the same | Canola) |
| | | | bacteria, and both lines contained the phosphinothricin N-acetyltransferase | |
| | | | (PAT) encoding gene from Streptomyces hygroscopicus. | |
| A-13 | MS1, RF2 | Aventis CropScience | Male-sterility, fertility restoration, pollination control system displaying | Brassica |
| | =>PGS2 | (formerly Plant | glufosinate herbicide tolerance. MS lines contained the barnase gene from | napus (Argentine |
| | | Genetic Systems) | Bacillus amyloliquefaciens, RF lines contained the barstar gene from the same | Canola) |
| | | | bacteria, and both lines contained the phosphinothricin N-acetyltransferase | |
| | | | (PAT) encoding gene from Streptomyces hygroscopicus. | |
| A-14 | MS8xRF3 | Bayer CropScience | Male-sterility, fertility restoration, pollination control system displaying | Brassica |
| | | (Aventis | glufosinate herbicide tolerance. MS lines contained the barnase gene from | napus (Argentine |
| | | CropScience(AgrEvo)) | Bacillus amyloliquefaciens, RF lines contained the barstar gene from the same | Canola) |
| | | | bacteria, and both lines contained the phosphinothricin N-acetyltransferase | |
| | | | (PAT) encoding gene from Streptomyces hygroscopicus. | |
| A-15 | NS738, | Pioneer Hi-Bred | Selection of somaclonal variants with altered acetolactate synthase (ALS) | Brassica |
| | NS1471, | International Inc. | enzymes, following chemical mutagenesis. Two lines (P1, P2) were initially | napus (Argentine |
| | NS1473 | | selected with modifications at different unlinked loci. NS738 contains the P2 | Canola) |
| | | | mutation only. | |
| A-16 | OXY-235 | Aventis CropScience | Tolerance to the herbicides bromoxynil and ioxynil by incorporation of the | Brassica |
| | | (formerly Rhône | nitrilase gene from Klebsiella pneumoniae. | napus (Argentine |
| | | Poulenc Inc.) | | Canola) |
| A-17 | PHY14, | Aventis CropScience | Male sterility was via insertion of the barnase ribonuclease gene from Bacillus | Brassica |
| | PHY35 | (formerly Plant | amyloliquefaciens; fertility restoration by insertion of the barstar RNase | napus (Argentine |
| | | Genetic Systems) | inhibitor; PPT resistance was via PPT-acetyltransferase (PAT) from | Canola) |
| | | | Streptomyces hygroscopicus. | |
| A-18 | PHY36 | Aventis CropScience | Male sterility was via insertion of the barnase ribonuclease gene from Bacillus | Brassica |
| | | (formerly Plant | amyloliquefaciens; fertility restoration by insertion of the barstar RNase | napus (Argentine |
| | | Genetic Systems) | inhibitor; PPT resistance was via PPT-acetyltransferase (PAT) from | Canola) |
| | | | Streptomyces hygroscopicus. | |
| A-19 | T45 | Bayer CropScience | Introduction of the PPT-acetyltransferase (PAT) encoding gene from | Brassica |
| | (HCN28) | (Aventis | Streptomyces viridochromogenes, an aerobic soil bacteria. PPT normally acts | napus (Argentine |
| | | CropScience(AgrEvo)) | to inhibit glutamine synthetase, causing a fatal accumulation of ammonia. | Canola) |
| | | | Acetylated PPT is inactive. | |
| A-20 | HCR-1 | Bayer CropScience | Introduction of the glufosinate ammonium herbicide tolerance trait from | Brassica rapa (Polish |
| | | (Aventis | transgenic B. napus line T45. This trait is mediated by the phosphinothricin | Canola) |
| | | CropScience(AgrEvo)) | acetyltransferase (PAT) encoding gene from S. viridochromogenes. | |
| A-21 | ZSR500/502 | Monsanto Company | Introduction of a modified 5-enol-pyruvylshikimate-3-phosphate synthase | Brassica rapa (Polish |
| | | | (EPSPS) and a gene from Achromobacter sp that degrades glyphosate by | Canola) |
| | | | conversion to aminomethylphosphonic acid (AMPA) and glyoxylate by | |
| | | | interspecific crossing with GT73. | |
| A-22 | 55-1/63-1 | Cornell University | Papaya ringspot virus (PRSV) resistant papaya produced by inserting the coat | Carica |
| | | | protein (CP) encoding sequences from this plant potyvirus. | papaya (Papaya) |
| A-23 | RM3-3, | Bejo Zaden BV | Male sterility was via insertion of the barnase ribonuclease gene from Bacillus | Cichorium |
| | RM3-4, | | amyloliquefaciens; PPT resistance was via the bar gene from S. hygroscopicus, | intybus (Chicory) |
| | RM3-6 | | which encodes the PAT enzyme. | |
| A-24 | A, B | Agritope Inc. | Reduced accumulation of S-adenosylmethionine (SAM), and consequently | Cucumis |
| | | | reduced ethylene synthesis, by introduction of the gene encoding S- | melo (Melon) |
| | | | adenosylmethionine hydrolase. | |
| A-25 | CZW-3 | Asgrow (USA); | Cucumber mosiac virus (CMV), zucchini yellows mosaic (ZYMV) and | Cucurbita |
| | | Seminis Vegetable | watermelon mosaic virus (WMV) 2 resistant squash (Curcurbita pepo) | pepo (Squash) |
| | | Inc. (Canada) | produced by inserting the coat protein (CP) encoding sequences from each of | |
| | | | these plant viruses into the host genome. | |
| A-26 | ZW20 | Upjohn (USA); | Zucchini yellows mosaic (ZYMV) and watermelon mosaic virus (WMV) 2 | Cucurbita |
| | | Seminis Vegetable | resistant squash (Curcurbita pepo) produced by inserting the coat protein (CP) | pepo (Squash) |
| | | Inc. (Canada) | encoding sequences from each of these plant potyviruses into the host genome. | |
| A-27 | 66 | Florigene Pty Ltd. | Delayed senescence and sulfonylurea herbicide tolerant carnations produced | Dianthus |
| | | | by inserting a truncated copy of the carnation aminocyclopropane cyclase | caryophyllus |
| | | | (ACC) synthase encoding gene in order to suppress expression of the | (Carnation) |
| | | | endogenous unmodified gene, which is required for normal ethylene | |
| | | | biosynthesis. Tolerance to sulfonyl urea herbicides was via the introduction of | |
| | | | a chlorsulfuron tolerant version of the acetolactate synthase (ALS) encoding | |
| | | | gene from tobacco. | |
| A-28 | 4, 11, 15, | Florigene Pty Ltd. | Modified colour and sulfonylurea herbicide tolerant carnations produced by | Dianthus |
| | 16 | | inserting two anthocyanin biosynthetic genes whose expression results in a | caryophyllus |
| | | | violet/mauve colouration. Tolerance to sulfonyl urea herbicides was via the | (Carnation) |
| | | | introduction of a chlorsulfuron tolerant version of the acetolactate synthase | |
| | | | (ALS) encoding gene from tobacco. | |
| A-29 | 959A, | Florigene Pty Ltd. | Introduction of two anthocyanin biosynthetic genes to result in a violet/mauve | Dianthus |
| | 988A, | | colouration; Introduction of a variant form of acetolactate synthase (ALS). | caryophyllus |
| | 1226A, | | | (Carnation) |
| | 1351A, | | | |
| | 1363A, | | | |
| | 1400A | | | |
| A-30 | A2704- | Aventis CropScience | Glufosinate ammonium herbicide tolerant soybean produced by inserting a | Glycine max |
| | 12, | | modified phosphinothricin acetyltransferase (PAT) encoding gene from the | L. (Soybean) |
| | A2704- | | soil bacterium Streptomyces viridochromogenes. | |
| | 21, | | | |
| | A5547-35 | | | |
| A-31 | A5547- | Bayer CropScience | Glufosinate ammonium herbicide tolerant soybean produced by inserting a | Glycine max |
| | 127 | (Aventis | modified phosphinothricin acetyltransferase (PAT) encoding gene from the | L. (Soybean) |
| | | CropScience(AgrEvo)) | soil bacterium Streptomyces viridochromogenes. | |
| A-32 | DP356043 | Pioneer Hi-Bred | Soybean event with two herbicide tolerance genes: glyphosate N- | Glycine max |
| | | International Inc. | acetlytransferase, which detoxifies glyphosate, and a modified acetolactate | L. (Soybean) |
| | | | synthase (A | |
| A-33 | G94-1, | DuPont Canada | High oleic acid soybean produced by inserting a second copy of the fatty acid | Glycine max |
| | G94-19, | Agricultural Products | desaturase (GmFad2-1) encoding gene from soybean, which resulted in | L. (Soybean) |
| | G168 | | “silencing” of the endogenous host gene. | |
| A-34 | GTS 40- | Monsanto Company | Glyphosate tolerant soybean variety produced by inserting a modified 5- | Glycine max |
| | 3-2 | | enolpyruvylshikimate-3-phosphate synthase (EPSPS) encoding gene from the | L. (Soybean) |
| | | | soil bacterium Agrobacterium tumefaciens. | |
| A-35 | GU262 | Bayer CropScience | Glufosinate ammonium herbicide tolerant soybean produced by inserting a | Glycine max |
| | | (Aventis | modified phosphinothricin acetyltransferase (PAT) encoding gene from the | L. (Soybean) |
| | | CropScience(AgrEvo)) | soil bacterium Streptomyces viridochromogenes. | |
| A-36 | MON89788 | Monsanto Company | Glyphosate-tolerant soybean produced by inserting a modified 5- | Glycine max |
| | | | enolpyruvylshikimate-3-phosphate synthase (EPSPS) encoding aroA (epsps) | L. (Soybean) |
| | | | gene from Agrobacterium tumefaciens CP4. | |
| A-37 | OT96-15 | Agriculture & | Low linolenic acid soybean produced through traditional cross-breeding to | Glycine max |
| | | Agri-Food Canada | incorporate the novel trait from a naturally occurring fan1 gene mutant that | L. (Soybean) |
| | | | was selected for low linolenic acid. | |
| A-38 | W62, | Bayer CropScience | Glufosinate ammonium herbicide tolerant soybean produced by inserting a | Glycine max |
| | W98 | (Aventis | modified phosphinothricin acetyltransferase (PAT) encoding gene from the | L. (Soybean) |
| | | CropScience(AgrEvo)) | soil bacterium Streptomyces hygroscopicus. | |
| A-39 | 15985 | Monsanto Company | Insect resistant cotton derived by transformation of the DP50B parent variety, | Gossypium hirsutum |
| | | | which contained event 531 (expressing Cry1Ac protein), with purified plasmid | L. (Cotton) |
| | | | DNA containing the cry2Ab gene from B. thuringiensis subsp. kurstaki. | |
| A-40 | 19-51A | DuPont Canada | Introduction of a variant form of acetolactate synthase (ALS). | Gossypium hirsutum |
| | | Agricultural Products | | L. (Cotton) |
| A-41 | 281-24- | DOW AgroSciences | Insect-resistant cotton produced by inserting the cry1F gene from Bacillus | Gossypium hirsutum |
| | 236 | LLC | thuringiensis var. aizawai. The PAT encoding gene from Streptomyces | L. (Cotton) |
| | | | viridochromogenes was introduced as a selectable marker. | |
| A-42 | 3006-210- | DOW AgroSciences | Insect-resistant cotton produced by inserting the cry1Ac gene from Bacillus | Gossypium hirsutum |
| | 23 | LLC | thuringiensis subsp. kurstaki. The PAT encoding gene from Streptomyces | L. (Cotton) |
| | | | viridochromogenes was introduced as a selectable marker. | |
| A-43 | 31807/31808 | Calgene Inc. | Insect-resistant and bromoxynil herbicide tolerant cotton produced by inserting | Gossypium hirsutum |
| | | | the cry1Ac gene from Bacillus thuringiensis and a nitrilase encoding gene | L. (Cotton) |
| | | | from Klebsiella pneumoniae. | |
| A-44 | BXN | Calgene Inc. | Bromoxynil herbicide tolerant cotton produced by inserting a nitrilase | Gossypium hirsutum |
| | | | encoding gene from Klebsiella pneumoniae. | L. (Cotton) |
| A-45 | COT102 | Syngenta Seeds, Inc. | Insect-resistant cotton produced by inserting the vip3A(a) gene from Bacillus | Gossypium hirsutum |
| | | | thuringiensisAB88. The APH4 encoding gene from E. coli was introduced as a | L. (Cotton) |
| | | | selectable marker. | |
| A-46 | DAS- | DOW AgroSciences | WideStrike ™, a stacked insect-resistant cotton derived from conventional | Gossypium hirsutum |
| | 21Ø23-5 x | LLC | cross-breeding of parental lines 3006-210-23 (OECD identifier: DAS-21Ø23- | L. (Cotton) |
| | DAS- | | 5) and 281-24-236 (OECD identifier: DAS-24236-5). | |
| | 24236-5 | | | |
| A-47 | DAS- | DOW AgroSciences | Stacked insect-resistant and glyphosate-tolerant cotton derived from | Gossypium hirsutum |
| | 21Ø23-5 x | LLC and Pioneer | conventional cross-breeding of WideStrike cotton (OECD identifier: DAS- | L. (Cotton) |
| | DAS- | Hi-Bred | 21Ø23-5 x DAS-24236-5) with MON88913, known as RoundupReady Flex | |
| | 24236-5 x | International Inc. | (OECD identifier: MON-88913-8). | |
| | MON88913 | | | |
| A-48 | DAS- | DOW AgroSciences | WideStrike ™/Roundup Ready ® cotton, a stacked insect-resistant and | Gossypium hirsutum |
| | 21Ø23-5 x | LLC | glyphosate-tolerant cotton derived from conventional cross-breeding of | L. (Cotton) |
| | DAS- | | WideStrike cotton (OECD identifier: DAS-21Ø23-5 x DAS-24236-5) with | |
| | 24236-5 x | | MON1445 (OECD identifier: MON-Ø1445-2). | |
| | MON- | | | |
| | Ø1445-2 | | | |
| A-49 | LLCotton | Bayer CropScience | Glufosinate ammonium herbicide tolerant cotton produced by inserting a | Gossypium hirsutum |
| | 25 | (Aventis | modified phosphinothricin acetyltransferase (PAT) encoding gene from the | L. (Cotton) |
| | | CropScience(AgrEvo)) | soil bacterium Streptomyces hygroscopicus. | |
| A-50 | LLCotton | Bayer CropScience | Stacked herbicide tolerant and insect resistant cotton combining tolerance to | Gossypium hirsutum |
| | 25 x | (Aventis | glufosinate ammonium herbicide from LLCotton25 (OECD identifier: ACS- | L. (Cotton) |
| | MON15985 | CropScience(AgrEvo)) | GHØØ1-3) with resistance to insects from MON15985 (OECD identifier: | |
| | | | MON-15985-7) | |
| A-51 | MON1445/ | Monsanto Company | Glyphosate herbicide tolerant cotton produced by inserting a naturally | Gossypium hirsutum |
| | 1698 | | glyphosate tolerant form of the enzyme 5-enolpyruvyl shikimate-3-phosphate | L. (Cotton) |
| | | | synthase (EPSPS) from A. tumefaciens strain CP4. | |
| A-52 | MON15985 x | Monsanto Company | Stacked insect resistant and glyphosate tolerant cotton produced by | Gossypium hirsutum |
| | MON88913 | | conventional cross-breeding of the parental lines MON88913 (OECD | L. (Cotton) |
| | | | identifier: MON-88913-8) and 15985 (OECD identifier: MON-15985-7). | |
| | | | Glyphosate tolerance is derived from MON88913 which contains two genes | |
| | | | encoding the enzyme 5-enolypyruvylshikimate-3-phosphate synthase (EPSPS) | |
| | | | from the CP4 strain of Agrobacterium tumefaciens. Insect resistance is derived | |
| | | | MON15985 which was produced by transformation of the DP50B parent | |
| | | | variety, which contained event 531 (expressing Cry1Ac protein), with purified | |
| | | | plasmid DNA containing the cry2Ab gene from B. thuringiensis subsp. | |
| | | | kurstaki. | |
| A-53 | MON- | Monsanto Company | Stacked insect resistant and herbicide tolerant cotton derived from | Gossypium hirsutum |
| | 15985-7 x | | conventional cross-breeding of the parental lines 15985 (OECD identifier: | L. (Cotton) |
| | MON- | | MON-15985-7) and MON1445 (OECD identifier: MON-Ø1445-2). | |
| | Ø1445-2 | | | |
| A-54 | MON531/ | Monsanto Company | Insect-resistant cotton produced by inserting the cry1Ac gene from Bacillus | Gossypium hirsutum |
| | 757/1076 | | thuringiensis subsp. kurstaki HD-73 (B.t.k.). | L. (Cotton) |
| A-55 | MON88913 | Monsanto Company | Glyphosate herbicide tolerant cotton produced by inserting two genes encoding | Gossypium hirsutum |
| | | | the enzyme 5-enolypyruvylshikimate-3-phosphate synthase (EPSPS) from the | L. (Cotton) |
| | | | CP4 strain of Agrobacterium tumefaciens. | |
| A-56 | MON- | Monsanto Company | Stacked insect resistant and herbicide tolerant cotton derived from | Gossypium hirsutum |
| | ØØ531-6 x | | conventional cross-breeding of the parental lines MON531 (OECD identifier: | L. (Cotton) |
| | MON- | | MON-ØØ531-6) and MON1445 (OECD identifier: MON-Ø1445-2). | |
| | Ø1445-2 | | | |
| A-57 | X81359 | BASF Inc. | Tolerance to imidazolinone herbicides by selection of a naturally occurring | Helianthus |
| | | | mutant. | annuus (Sunflower) |
| A-58 | RH44 | BASF Inc. | Selection for a mutagenized version of the enzyme acetohydroxyacid synthase | Lens |
| | | | (AHAS), also known as acetolactate synthase (ALS) or acetolactate pyruvate- | culinaris (Lentil) |
| | | | lyase. | |
| A-59 | FP967 | University of | A variant form of acetolactate synthase (ALS) was obtained from a | Linum usitatissimum |
| | | Saskatchewan, Crop | chlorsulfuron tolerant line of A. thaliana and used to transform flax. | L. (Flax, Linseed) |
| | | Dev. Centre | | |
| A-60 | 5345 | Monsanto Company | Resistance to lepidopteran pests through the introduction of the cry1Ac gene | Lycopersicon |
| | | | from Bacillus thuringiensis subsp. Kurstaki. | esculentum (Tomato) |
| A-61 | 8338 | Monsanto Company | Introduction of a gene sequence encoding the enzyme 1-amino-cyclopropane- | Lycopersicon |
| | | | 1-carboxylic acid deaminase (ACCd) that metabolizes the precursor of the fruit | esculentum (Tomato) |
| | | | ripening hormone ethylene. | |
| A-62 | 1345-4 | DNA Plant | Delayed ripening tomatoes produced by inserting an additional copy of a | Lycopersicon |
| | | Technology | truncated gene encoding 1-aminocyclopropane-1-carboxyllic acid (ACC) | esculentum (Tomato) |
| | | Corporation | synthase, which resulted in downregulation of the endogenous ACC synthase | |
| | | | and reduced ethylene accumulation. | |
| A-63 | 35 1 N | Agritope Inc. | Introduction of a gene sequence encoding the enzyme S-adenosylmethionine | Lycopersicon |
| | | | hydrolase that metabolizes the precursor of the fruit ripening hormone ethylene | esculentum (Tomato) |
| A-64 | B, Da, F | Zeneca Seeds | Delayed softening tomatoes produced by inserting a truncated version of the | Lycopersicon |
| | | | polygalacturonase (PG) encoding gene in the sense or anti-sense orientation in | esculentum (Tomato) |
| | | | order to reduce expression of the endogenous PG gene, and thus reduce pectin | |
| | | | degradation. | |
| A-65 | FLAVR | Calgene Inc. | Delayed softening tomatoes produced by inserting an additional copy of the | Lycopersicon |
| | SAVR | | polygalacturonase (PG) encoding gene in the anti-sense orientation in order to | esculentum (Tomato) |
| | | | reduce expression of the endogenous PG gene and thus reduce pectin | |
| | | | degradation. | |
| A-66 | J101, | Monsanto Company | Glyphosate herbicide tolerant alfalfa (lucerne) produced by inserting a gene | Medicago |
| | J163 | and Forage | encoding the enzyme 5-enolypyruvylshikimate-3-phosphate synthase (EPSPS) | sativa (Alfalfa) |
| | | Genetics | from the CP4 strain of Agrobacterium tumefaciens. | |
| | | International | | |
| A-67 | C/F/93/08- | Societe National | Tolerance to the herbicides bromoxynil and ioxynil by incorporation of the | Nicotiana tabacum |
| | 02 | d'Exploitation des | nitrilase gene from Klebsiella pneumoniae. | L. (Tobacco) |
| | | Tabacs et Allumettes | | |
| A-68 | Vector | Vector Tobacco Inc. | Reduced nicotine content through introduction of a second copy of the tobacco | Nicotiana tabacum |
| | 21-41 | | quinolinic acid phosphoribosyltransferase (QTPase) in the antisense | L. (Tobacco) |
| | | | orientation. The NPTII encoding gene from E. coli was introduced as a | |
| | | | selectable marker to identify transformants. | |
| A-69 | CL121, | BASF Inc. | Tolerance to the imidazolinone herbicide, imazethapyr, induced by chemical | Oryza sativa (Rice) |
| | CL141, | | mutagenesis of the acetolactate synthase (ALS) enzyme using ethyl | |
| | CFX51 | | methanesulfonate (EMS). | |
| A-70 | IMINTA- | BASF Inc. | Tolerance to imidazolinone herbicides induced by chemical mutagenesis of the | Oryza sativa (Rice) |
| | 1, | | acetolactate synthase (ALS) enzyme using sodium azide. | |
| | IMINTA-4 | | | |
| A-71 | LLRICE06, | Aventis CropScience | Glufosinate ammonium herbicide tolerant rice produced by inserting a | Oryza sativa (Rice) |
| | LLRICE62 | | modified phosphinothricin acetyltransferase (PAT) encoding gene from the | |
| | | | soil bacterium Streptomyces hygroscopicus). | |
| A-72 | LLRICE601 | Bayer CropScience | Glufosinate ammonium herbicide tolerant rice produced by inserting a | Oryza sativa (Rice) |
| | | (Aventis | modified phosphinothricin acetyltransferase (PAT) encoding gene from the | |
| | | CropScience(AgrEvo)) | soil bacterium Streptomyces hygroscopicus). | |
| A-73 | C5 | United States | Plum pox virus (PPV) resistant plum tree produced through Agrobacterium- | Prunus domestica |
| | | Department of | mediated transformation with a coat protein (CP) gene from the virus. | (Plum) |
| | | Agriculture - | | |
| | | Agricultural Research | | |
| | | Service | | |
| A-74 | PWC16 | BASF Inc. | Tolerance to the imidazolinone herbicide, imazethapyr, induced by chemical | Oryza sativa (Rice) |
| | | | mutagenesis of the acetolactate synthase (ALS) enzyme using ethyl | |
| | | | methanesulfonate (EMS). | |
| A-75 | ATBT04- | Monsanto Company | Colorado potato beetle resistant potatoes produced by inserting the cry3A gene | Solanum tuberosum |
| | 6, | | from Bacillus thuringiensis (subsp. Tenebrionis). | L. (Potato) |
| | ATBT04- | | | |
| | 27, | | | |
| | ATBT04- | | | |
| | 30, | | | |
| | ATBT04- | | | |
| | 31, | | | |
| | ATBT04- | | | |
| | 36, | | | |
| | SPBT02- | | | |
| | 5, | | | |
| | SPBT02-7 | | | |
| A-76 | BT6, | Monsanto Company | Colorado potato beetle resistant potatoes produced by inserting the cry3A gene | Solanum tuberosum |
| | BT10, | | from Bacillus thuringiensis (subsp. Tenebrionis). | L. (Potato) |
| | BT12, | | | |
| | BT16, | | | |
| | BT17, | | | |
| | BT18, | | | |
| | BT23 | | | |
| A-77 | RBMT15- | Monsanto Company | Colorado potato beetle and potato virus Y (PVY) resistant potatoes produced | Solanum tuberosum |
| | 101, | | by inserting the cry3A gene from Bacillus thuringiensis (subsp. Tenebrionis) | L. (Potato) |
| | SEMT15- | | and the coat protein encoding gene from PVY. | |
| | 02, | | | |
| | SEMT15- | | | |
| | 15 | | | |
| A-78 | RBMT21- | Monsanto Company | Colorado potato beetle and potato leafroll virus (PLRV) resistant potatoes | Solanum tuberosum |
| | 129, | | produced by inserting the cry3A gene from Bacillus thuringiensis (subsp. | L. (Potato) |
| | RBMT21- | | Tenebrionis) and the replicase encoding gene from PLRV. | |
| | 350, | | | |
| | RBMT22- | | | |
| | 082 | | | |
| A-79 | AP205CL | BASF Inc. | Selection for a mutagenized version of the enzyme acetohydroxyacid synthase | Triticum |
| | | | (AHAS), also known as acetolactate synthase (ALS) or acetolactate pyruvate- | aestivum (Wheat) |
| | | | lyase. | |
| A-80 | AP602CL | BASF Inc. | Selection for a mutagenized version of the enzyme acetohydroxyacid synthase | Triticum |
| | | | (AHAS), also known as acetolactate synthase (ALS) or acetolactate pyruvate- | aestivum (Wheat) |
| | | | lyase. | |
| A-81 | BW255-2, | BASF Inc. | Selection for a mutagenized version of the enzyme acetohydroxyacid synthase | Triticum |
| | BW238-3 | | (AHAS), also known as acetolactate synthase (ALS) or acetolactate pyruvate- | aestivum (Wheat) |
| | | | lyase. | |
| A-82 | BW7 | BASF Inc. | Tolerance to imidazolinone herbicides induced by chemical mutagenesis of the | Triticum |
| | | | acetohydroxyacid synthase (AHAS) gene using sodium azide. | aestivum (Wheat) |
| A-83 | MON71800 | Monsanto Company | Glyphosate tolerant wheat variety produced by inserting a modified 5- | Triticum |
| | | | enolpyruvylshikimate-3-phosphate synthase (EPSPS) encoding gene from the | aestivum (Wheat) |
| | | | soil bacterium Agrobacterium tumefaciens, strain CP4. | |
| A-84 | SWP965001 | Cyanamid Crop | Selection for a mutagenized version of the enzyme acetohydroxyacid synthase | Triticum |
| | | Protection | (AHAS), also known as acetolactate synthase (ALS) or acetolactate pyruvate- | aestivum (Wheat) |
| | | | lyase. | |
| A-85 | Teal 11A | BASF Inc. | Selection for a mutagenized version of the enzyme acetohydroxyacid synthase | Triticum |
| | | | (AHAS), also known as acetolactate synthase (ALS) or acetolactate pyruvate- | aestivum (Wheat) |
| | | | lyase. | |
| A-86 | 176 | Syngenta Seeds, Inc. | Insect-resistant maize produced by inserting the cry1Ab gene from Bacillus | Zea mays L. (Maize) |
| | | | thuringiensis subsp. kurstaki. The genetic modification affords resistance to | |
| | | | attack by the European corn borer (ECB). | |
| A-87 | 3751IR | Pioneer Hi-Bred | Selection of somaclonal variants by culture of embryos on imidazolinone | Zea mays L. (Maize) |
| | | International Inc. | containing media. | |
| A-88 | 676, 678, | Pioneer Hi-Bred | Male-sterile and glufosinate ammonium herbicide tolerant maize produced by | Zea mays L. (Maize) |
| | 680 | International Inc. | inserting genes encoding DNA adenine methylase and phosphinothricin | |
| | | | acetyltransferase (PAT) from Escherichia coli and Streptomyces | |
| | | | viridochromogenes, respectively. | |
| A-89 | ACS- | Bayer CropScience | Stacked insect resistant and herbicide tolerant corn hybrid derived from | Zea mays L. (Maize) |
| | ZMØØ3- | (Aventis | conventional cross-breeding of the parental lines T25 (OECD identifier: ACS- | |
| | 2 x MON- | CropScience(AgrEvo)) | ZMØØ3-2) and MON810 (OECD identifier: MON-ØØ81Ø-6). | |
| | ØØ81Ø-6 | | | |
| A-90 | B16 | Dekalb Genetics | Glufosinate ammonium herbicide tolerant maize produced by inserting the | Zea mays L. (Maize) |
| | (DLL25) | Corporation | gene encoding phosphinothricin acetyltransferase (PAT) from Streptomyces | |
| | | | hygroscopicus. | |
| A-91 | BT11 | Syngenta Seeds, Inc. | Insect-resistant and herbicide tolerant maize produced by inserting the cry1Ab | Zea mays L. (Maize) |
| | (X4334CBR, | | gene from Bacillus thuringiensis subsp. kurstaki, and the phosphinothricin N- | |
| | X4734CBR) | | acetyltransferase (PAT) encoding gene from S. viridochromogenes. | |
| A-92 | BT11 x | Syngenta Seeds, Inc. | Stacked insect resistant and herbicide tolerant maize produced by conventional | Zea mays L. (Maize) |
| | MIR604 | | cross breeding of parental lines BT11 (OECD unique identifier: SYN-BTØ11- | |
| | | | 1) and MIR604 (OECD unique identifier: SYN-IR6Ø5-5). Resistance to the | |
| | | | European Corn Borer and tolerance to the herbicide glufosinate ammonium | |
| | | | (Liberty) is derived from BT11, which contains the cry1Ab gene from Bacillus | |
| | | | thuringiensis subsp. kurstaki, and the phosphinothricin N-acetyltransferase | |
| | | | (PAT) encoding gene from S. viridochromogenes. Corn rootworm-resistance is | |
| | | | derived from MIR604 which contains the mcry3A gene from Bacillus | |
| | | | thuringiensis. | |
| A-93 | BT11 x | Syngenta Seeds, Inc. | Stacked insect resistant and herbicide tolerant maize produced by conventional | Zea mays L. (Maize) |
| | MIR604 x | | cross breeding of parental lines BT11 (OECD unique identifier: SYN-BTØ11- | |
| | GA21 | | 1), MIR604 (OECD unique identifier: SYN-IR6Ø5-5) and GA21 (OECD | |
| | | | unique identifier: MON-ØØØ21-9). Resistance to the European Corn Borer | |
| | | | and tolerance to the herbicide glufosinate ammonium (Liberty) is derived from | |
| | | | BT11, which contains the cry1Ab gene from Bacillus thuringiensis subsp. | |
| | | | kurstaki, and the phosphinothricin N-acetyltransferase (PAT) encoding gene | |
| | | | from S. viridochromogenes. Corn rootworm-resistance is derived from | |
| | | | MIR604 which contains the mcry3A gene from Bacillus thuringiensis. | |
| | | | Tolerance to glyphosate herbcicide is derived from GA21 which contains a a | |
| | | | modified EPSPS gene from maize. | |
| A-94 | CBH-351 | Aventis CropScience | Insect-resistant and glufosinate ammonium herbicide tolerant maize developed | Zea mays L. (Maize) |
| | | | by inserting genes encoding Cry9C protein from Bacillus thuringiensis subsp | |
| | | | tolworthi and phosphinothricin acetyltransferase (PAT) from Streptomyces | |
| | | | hygroscopicus. | |
| A-95 | DAS- | DOW AgroSciences | Lepidopteran insect resistant and glufosinate ammonium herbicide-tolerant | Zea mays L. (Maize) |
| | 06275-8 | LLC | maize variety produced by inserting the cry1F gene from Bacillus thuringiensis | |
| | | | var aizawai and the phosphinothricin acetyltransferase (PAT) from | |
| | | | Streptomyces hygroscopicus. | |
| A-96 | DAS- | DOW AgroSciences | Corn rootworm-resistant maize produced by inserting the cry34Ab1 and | Zea mays L. (Maize) |
| | 59122-7 | LLC and Pioneer | cry35Ab1 genes from Bacillus thuringiensis strain PS149B1. The PAT | |
| | | Hi-Bred International | encoding gene from Streptomyces viridochromogenes was introduced as a | |
| | | Inc. | selectable marker. | |
| A-97 | DAS- | DOW AgroSciences | Stacked insect resistant and herbicide tolerant maize produced by conventional | Zea mays L. (Maize) |
| | 59122-7 x | LLC and Pioneer | cross breeding of parental lines DAS-59122-7 (OECD unique identifier: DAS- | |
| | NK603 | Hi-Bred International | 59122-7) with NK603 (OECD unique identifier: MON-ØØ6Ø3-6). Corn | |
| | | Inc. | rootworm-resistance is derived from DAS-59122-7 which contains the | |
| | | | cry34Ab1 and cry35Ab1 genes from Bacillus thuringiensis strain PS149B1. | |
| | | | Tolerance to glyphosate herbcicide is derived from NK603. | |
| A-98 | DAS- | DOW AgroSciences | Stacked insect resistant and herbicide tolerant maize produced by conventional | Zea mays L. (Maize) |
| | 59122-7 x | LLC and Pioneer | cross breeding of parental lines DAS-59122-7 (OECD unique identifier: DAS- | |
| | TC1507 x | Hi-Bred International | 59122-7) and TC1507 (OECD unique identifier: DAS-Ø15Ø7-1) with NK603 | |
| | NK603 | Inc. | (OECD unique identifier: MON-ØØ6Ø3-6). Corn rootworm-resistance is | |
| | | | derived from DAS-59122-7 which contains the cry34Ab1 and cry35Ab1 genes | |
| | | | from Bacillus thuringiensis strain PS149B1. Lepidopteran resistance and | |
| | | | toleraance to glufosinate ammonium herbicide is derived from TC1507. | |
| | | | Tolerance to glyphosate herbcicide is derived from NK603. | |
| A-99 | DAS- | DOW AgroSciences | Stacked insect resistant and herbicide tolerant corn hybrid derived from | Zea mays L. (Maize) |
| | Ø15Ø7-1 x | LLC | conventional cross-breeding of the parental lines 1507 (OECD identifier: | |
| | MON- | | DAS-Ø15Ø7-1) and NK603 (OECD identifier: MON-ØØ6Ø3-6). | |
| | ØØ6Ø3-6 | | | |
| A- | DBT418 | Dekalb Genetics | Insect-resistant and glufosinate ammonium herbicide tolerant maize developed | Zea mays L. (Maize) |
| 100 | | Corporation | by inserting genes encoding Cry1AC protein from Bacillus thuringiensis subsp | |
| | | | kurstaki and phosphinothricin acetyltransferase (PAT) from Streptomyces | |
| | | | hygroscopicus | |
| A- | DK404SR | BASF Inc. | Somaclonal variants with a modified acetyl-CoA-carboxylase (ACCase) were | Zea mays L. (Maize) |
| 101 | | | selected by culture of embryos on sethoxydim enriched medium. | |
| A- | Event | Syngenta Seeds, Inc. | Maize line expressing a heat stable alpha-amylase gene amy797E for use in the | Zea mays L. (Maize) |
| 102 | 3272 | | dry-grind ethanol process. The phosphomannose isomerase gene from E. coli | |
| | | | was used as a selectable marker. | |
| A- | EXP1910 | Syngenta Seeds, Inc. | Tolerance to the imidazolinone herbicide, imazethapyr, induced by chemical | Zea mays L. (Maize) |
| 103 | IT | (formerly Zeneca | mutagenesis of the acetolactate synthase (ALS) enzyme using ethyl | |
| | | Seeds) | methanesulfonate (EMS). | |
| A- | GA21 | Monsanto Company | Introduction, by particle bombardment, of a modified 5-enolpyruvyl | Zea mays L. (Maize) |
| 104 | | | shikimate-3-phosphate synthase (EPSPS), an enzyme involved in the shikimate | |
| | | | biochemical pathway for the production of the aromatic amino acids. | |
| A- | IT | Pioneer Hi-Bred | Tolerance to the imidazolinone herbicide, imazethapyr, was obtained by in | Zea mays L. (Maize) |
| 105 | | International Inc. | vitro selection of somaclonal variants. | |
| A- | LY038 | Monsanto Company | Altered amino acid composition, specifically elevated levels of lysine, through | Zea mays L. (Maize) |
| 106 | | | the introduction of the cordapA gene, derived from Corynebacterium | |
| | | | glutamicum, encoding the enzyme dihydrodipicolinate synthase (cDHDPS). | |
| A- | MIR604 | Syngenta Seeds, Inc. | Corn rootworm resistant maize produced by transformation with a modified | Zea mays L. (Maize) |
| 107 | | | cry3A gene. The phosphomannose isomerase gene from E. coli was used as a | |
| | | | selectable marker. | |
| A- | MIR604 x | Syngenta Seeds, Inc. | Stacked insect resistant and herbicide tolerant maize produced by conventional | Zea mays L. (Maize) |
| 108 | GA21 | | cross breeding of parental lines MIR604 (OECD unique identifier: SYN- | |
| | | | IR6Ø5-5) and GA21 (OECD unique identifier: MON-ØØØ21-9). Corn | |
| | | | rootworm-resistance is derived from MIR604 which contains the mcry3A gene | |
| | | | from Bacillus thuringiensis. Tolerance to glyphosate herbcicide is derived | |
| | | | from GA21. | |
| A- | MON80100 | Monsanto Company | Insect-resistant maize produced by inserting the cry1Ab gene from Bacillus | Zea mays L. (Maize) |
| 109 | | | thuringiensis subsp. kurstaki. The genetic modification affords resistance to | |
| | | | attack by the European corn borer (ECB). | |
| A- | MON802 | Monsanto Company | Insect-resistant and glyphosate herbicide tolerant maize produced by inserting | Zea mays L. (Maize) |
| 110 | | | the genes encoding the Cry1Ab protein from Bacillus thuringiensis and the 5- | |
| | | | enolpyruvylshikimate-3-phosphate synthase (EPSPS) from A. tumefaciens | |
| | | | strain CP4. | |
| A- | MON809 | Pioneer Hi-Bred | Resistance to European corn borer (Ostrinia nubilalis) by introduction of a | Zea mays L. (Maize) |
| 111 | | International Inc. | synthetic cry1Ab gene. Glyphosate resistance via introduction of the bacterial | |
| | | | version of a plant enzyme, 5-enolpyruvyl shikimate-3-phosphate synthase | |
| | | | (EPSPS). | |
| A- | MON810 | Monsanto Company | Insect-resistant maize produced by inserting a truncated form of the cry1Ab | Zea mays L. (Maize) |
| 112 | | | gene from Bacillus thuringiensis subsp. kurstaki HD-1. The genetic | |
| | | | modification affords resistance to attack by the European corn borer (ECB). | |
| A- | MON810 x | Monsanto Company | Stacked insect resistant and glyphosate tolerant maize derived from | Zea mays L. (Maize) |
| 113 | MON88017 | | conventional cross-breeding of the parental lines MON810 (OECD identifier: | |
| | | | MON-ØØ81Ø-6) and MON88017 (OECD identifier: MON-88Ø17-3). | |
| | | | European corn borer (ECB) resistance is derived from a truncated form of the | |
| | | | cry1Ab gene from Bacillus thuringiensis subsp. kurstaki HD-1 present in | |
| | | | MON810. Corn rootworm resistance is derived from the cry3Bb1 gene from | |
| | | | Bacillus thuringiensis subspecies kumamotoensis strain EG4691 present in | |
| | | | MON88017. Glyphosate tolerance is derived from a 5-enolpyruvylshikimate- | |
| | | | 3-phosphate synthase (EPSPS) encoding gene from Agrobacterium | |
| | | | tumefaciens strain CP4 present in MON88017. | |
| A- | MON832 | Monsanto Company | Introduction, by particle bombardment, of glyphosate oxidase (GOX) and a | Zea mays L. (Maize) |
| 114 | | | modified 5-enolpyruvyl shikimate-3-phosphate synthase (EPSPS), an enzyme | |
| | | | involved in the shikimate biochemical pathway for the production of the | |
| | | | aromatic amino acids. | |
| A- | MON863 | Monsanto Company | Corn root worm resistant maize produced by inserting the cry3Bb1 gene from | Zea mays L. (Maize) |
| 115 | | | Bacillus thuringiensis subsp. kumamotoensis. | |
| A- | MON88017 | Monsanto Company | Corn rootworm-resistant maize produced by inserting the cry3Bb1 gene from | Zea mays L. (Maize) |
| 116 | | | Bacillus thuringiensis subspecies kumamotoensis strain EG4691. Glyphosate | |
| | | | tolerance derived by inserting a 5-enolpyruvylshikimate-3-phosphate synthase | |
| | | | (EPSPS) encoding gene from Agrobacterium tumefaciens strain CP4. | |
| A- | MON89034 | Monsanto Company | Maize event expressing two different insecticidal proteins from Bacillus | Zea mays L. (Maize) |
| 117 | | | thuringiensis providing resistance to number of lepidopteran pests. | |
| A- | MON89034 x | Monsanto Company | Stacked insect resistant and glyphosate tolerant maize derived from | Zea mays L. (Maize) |
| 118 | MON88017 | | conventional cross-breeding of the parental lines MON89034 (OECD | |
| | | | identifier: MON-89Ø34-3) and MON88017 (OECD identifier: MON-88Ø17- | |
| | | | 3). Resistance to Lepiopteran insects is derived from two crygenes present in | |
| | | | MON89043. Corn rootworm resistance is derived from a single cry genes and | |
| | | | glyphosate tolerance is derived from the 5-enolpyruvylshikimate-3-phosphate | |
| | | | synthase (EPSPS) encoding gene from Agrobacterium tumefaciens present in | |
| | | | MON88017. | |
| A- | MON- | Monsanto Company | Stacked insect resistant and herbicide tolerant corn hybrid derived from | Zea mays L. (Maize) |
| 119 | ØØ6Ø3-6 x | | conventional cross-breeding of the parental lines NK603 (OECD identifier: | |
| | MON- | | MON-ØØ6Ø3-6) and MON810 (OECD identifier: MON-ØØ81Ø-6). | |
| | ØØ81Ø-6 | | | |
| A- | MON- | Monsanto Company | Stacked insect resistant and enhanced lysine content maize derived from | Zea mays L. (Maize) |
| 120 | ØØ81Ø-6 x | | conventional cross-breeding of the parental lines MON810 (OECD identifier: | |
| | LY038 | | MON-ØØ81Ø-6) and LY038 (OECD identifier: REN-ØØØ38-3). | |
| A- | MON- | Monsanto Company | Stacked insect resistant and herbicide tolerant corn hybrid derived from | Zea mays L. (Maize) |
| 121 | ØØ863-5 x | | conventional cross-breeding of the parental lines MON863 (OECD | |
| | MON- | | identifier: MON-ØØ863-5) and NK603 (OECD identifier: MON-ØØ6Ø3-6). | |
| | ØØ6Ø3-6 | | | |
| A- | MON- | Monsanto Company | Stacked insect resistant corn hybrid derived from conventional cross-breeding | Zea mays L. (Maize) |
| 122 | ØØ863-5 x | | of the parental lines MON863 (OECD identifier: MON-ØØ863-5) and | |
| | MON- | | MON810 (OECD identifier: MON-ØØ81Ø-6) | |
| | ØØ863-5 | | | |
| A- | MON- | Monsanto Company | Stacked insect resistant and herbicide tolerant corn hybrid derived from | Zea mays L. (Maize) |
| 123 | ØØ863-5 x | | conventional cross-breeding of the stacked hybrid MON-ØØ863-5 x MON- | |
| | MON- | | ØØ81Ø-6 and NK603 (OECD identifier: MON-ØØ6Ø3-6). | |
| | ØØ81Ø-6 x | | | |
| | MON- | | | |
| | ØØ6Ø3-6 | | | |
| A- | MON- | Monsanto Company | Stacked insect resistant and herbicide tolerant corn hybrid derived from | Zea mays L. (Maize) |
| 124 | ØØØ21-9 x | | conventional cross-breeding of the parental lines GA21 (OECD identifider: | |
| | MON- | | MON-ØØØ21-9) and MON810 (OECD identifier: MON-ØØ81Ø-6). | |
| | ØØ81Ø-6 | | | |
| A- | MS3 | Bayer CropScience | Male sterility caused by expression of the barnase ribonuclease gene from | Zea mays L. (Maize) |
| 125 | | (Aventis | Bacillus amyloliquefaciens; PPT resistance was via PPT-acetyltransferase | |
| | | CropScience(AgrEvo)) | (PAT). | |
| A- | MS6 | Bayer CropScience | Male sterility caused by expression of the barnase ribonuclease gene from | Zea mays L. (Maize) |
| 126 | | (Aventis | Bacillus amyloliquefaciens; PPT resistance was via PPT-acetyltransferase | |
| | | CropScience(AgrEvo)) | (PAT). | |
| A- | NK603 | Monsanto Company | Introduction, by particle bombardment, of a modified 5-enolpyruvyl | Zea mays L. (Maize) |
| 127 | | | shikimate-3-phosphate synthase (EPSPS), an enzyme involved in the shikimate | |
| | | | biochemical pathway for the production of the aromatic amino acids. | |
| A- | SYN- | Syngenta Seeds, Inc. | Stacked insect resistant and herbicide tolerant maize produced by conventional | Zea mays L. (Maize) |
| 128 | BTØ11-1 x | | cross breeding of parental lines BT11 (OECD unique identifier: SYN-BTØ11- | |
| | MON- | | 1) and GA21 (OECD unique identifier: MON-ØØØ21-9). | |
| | ØØØ21-9 | | | |
| A- | T14, T25 | Bayer CropScience | Glufosinate herbicide tolerant maize produced by inserting the | Zea mays L. (Maize) |
| 129 | | (Aventis | phosphinothricin N-acetyltransferase (PAT) encoding gene from the aerobic | |
| | | CropScience(AgrEvo)) | actinomycete Streptomyces viridochromogenes. | |
| A- | TC1507 | Mycogen (c/o Dow | Insect-resistant and glufosinate ammonium herbicide tolerant maize produced | Zea mays L. (Maize) |
| 130 | | AgroSciences); | by inserting the cry1F gene from Bacillus thuringiensis var. aizawai and the | |
| | | Pioneer (c/o | phosphinothricin N-acetyltransferase encoding gene from Streptomyces | |
| | | Dupont) | viridochromogenes. | |
| A- | TC1507 x | DOW AgroSciences | Stacked insect resistant and herbicide tolerant maize produced by conventional | Zea mays L. (Maize) |
| 131 | DAS- | LLC and Pioneer | cross breeding of parental lines TC1507 (OECD unique identifier: DAS- | |
| | 59122-7 | Hi-Bred International | Ø15Ø7-1) with DAS-59122-7 (OECD unique identifier: DAS-59122-7). | |
| | | Inc. | Resistance to lepidopteran insects is derived from TC1507 due the presence of | |
| | | | the cry1F gene from Bacillus thuringiensis var. aizawai. Corn rootworm- | |
| | | | resistance is derived from DAS-59122-7 which contains the cry34Ab1 and | |
| | | | cry35Ab1 genes from Bacillus thuringiensis strain PS149B1. Tolerance to | |
| | | | glufosinate ammonium herbcicide is derived from TC1507 from the | |
| | | | phosphinothricin N-acetyltransferase encoding gene from Streptomyces | |
| | | | viridochromogenes. | |
| A- | MON89788 | Monsanto | Glyphosate-tolerant soybean produced by inserting a modified 5- | Soybean |
| 132 | | | enolpyruvylshikimate-3-phosphate synthase (EPSPS) encoding aroA (epsps) | |
| | | | gene from Agrobacterium tumefaciens CP4. | |

When used in the methods of the invention, the compounds of formula I may be in unmodified form or, preferably, formulated together with carriers and adjuvants conventionally employed in the art of formulation.

The invention therefore also relates to a composition for the control of mycotoxin contamination comprising a compound of formula (I) as defined above and an agriculturally acceptable support, carrier or filler.

According to the invention, the term “support” denotes a natural or synthetic, organic or inorganic compound with which the active compound of formula (I) is combined or associated to make it easier to apply, notably to the parts of the plant. This support is thus generally inert and should be agriculturally acceptable. The support may be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports may also be used.

The composition according to the invention may also comprise additional components. In particular, the composition may further comprise a surfactant. The surfactant can be an emulsifier, a dispersing agent or a wetting agent of ionic or non-ionic type or a mixture of such surfactants. Mention may be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the present compounds containing sulphate, sulphonate and phosphate functions. The presence of at least one surfactant is generally essential when the active compound and/or the inert support are water-insoluble and when the vector agent for the application is water. Preferably, surfactant content may be comprised from 5% to 40% by weight of the composition.

Colouring agents such as inorganic pigments, for example iron oxide, titanium oxide, ferrocyanblue, and organic pigments such as alizarin, azo and metallophthalocyanine dyes, and trace elements such as iron, manganese, boron, copper, cobalt, molybdenum and zinc salts can be used.

Optionally, other additional components may also be included, e.g. protective colloids, adhesives, thickeners, thixotropic agents, penetration agents, stabilisers, sequestering agents. More generally, the active compounds can be combined with any solid or liquid additive, which complies with the usual formulation techniques.

In general, the composition according to the invention may contain from 0.05 to 99% by weight of active compounds, preferably from 10 to 70% by weight.

The compounds or compositions according to the invention can be used as such, in form of their formulations or as the use forms prepared therefrom, such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder.

The treatment of plants and plant parts with the compounds or compositions according to the invention is carried out directly or by action on their environment, habitat or storage area by means of the normal treatment methods, for example by watering (drenching), drip irrigation, spraying, atomizing, broadcasting, dusting, foaming, spreading-on, and as a powder for dry seed treatment, a solution for seed treatment, a water-soluble powder for seed treatment, a water-soluble powder for slurry treatment, or by encrusting.

These compositions include not only compositions which are ready to be applied to the plant or seed to be treated by means of a suitable device, such as a spraying or dusting device, but also concentrated commercial compositions which must be diluted before application to the crop.

The compounds or compositions according to the invention can be employed for reducing mycotoxin contamination in crop protection or in the protection of materials.

Within the composition according to the invention, bactericide compounds can be employed in crop protection for example for controlling Pseudomonadaceae, Rhizobiaceae, Enterobacteriaceae, Corynebacteriaceae and Streptomycetaceae.

The compounds or compositions according to the invention can be used to curatively or preventively reduce the mycotoxin contamination of plants or crops. Thus, according to a further aspect of the invention, there is provided a method for curatively or preventively reduce the mycotoxin contamination of comprising the use of a composition comprising a compound according to formula (I) according to the invention by application to the seed, the plant or to the fruit of the plant or to the soil in which the plant is growing or in which it is desired to grow.

Suitably, the active ingredient may be applied to plant propagation material to be protected by impregnating the plant propagation material, in particular, seeds, either with a liquid formulation of the fungicide or coating it with a solid formulation. In special cases, other types of application are also possible, for example, the specific treatment of plant cuttings or twigs serving propagation.

The present invention will now be described by way of the following non-limiting examples.

### EXAMPLES

| Example No | Chemical Structure |
|---|---|
| 1 | 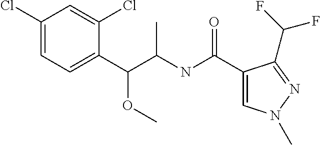 [SMILES: COC(c1ccc(Cl)cc1Cl)C(C)NC(=O)c1cn(C)nc1C(F)F] |
| 2 | 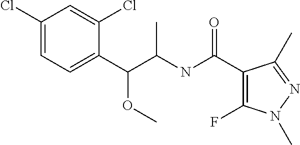 [SMILES: COC(c1ccc(Cl)cc1Cl)C(C)NC(=O)c1c(C)nn(C)c1F] |
| 3 | 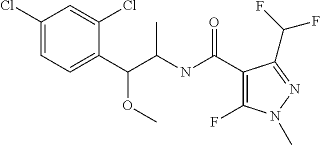 [SMILES: COC(c1ccc(Cl)cc1Cl)C(C)NC(=O)c1c(C(F)F)nn(C)c1F] |

Production of Fumonisin FB1 by Fusarlum proliferatum

Compounds were tested in microtiter plates in fumonisin-inducing liquid media (0.5 g malt extract, 1 g yeast extract, Ig bacto peptone, 20g Fructose, Ig KH₂PO₄, 0.3 g MgSO₄x7H₂O, 0.3 g KCl, 0.05 g ZnSO₄x7H₂O and 0.01 g CuSO₄x5H₂O per liter) containing 0.5% DMSO, inoculated with a concentrated spore suspension of Fusarium proliferatum to a final concentration of 2000 spores/ml.

Plates were covered and incubated at high humidity at 20° C. for 5 days

At start and after 5 days OD measurement at OD620 multiple read per well (square: 3×3) was taken to calculate growth inhibition.

After 5 days samples of each culture medium were taken and diluted 1:1000 in 50% acetonitrile.

The amounts of fumonisin FB1 of the samples were analysed per HPLC-MS/MS and results were used to calculate inhibition of FB1 production in comparison to a control without compound.

### Examples for Inhibition of Fumonisin FB1 Production

Compounds listed below showed an activity of >80% of inhibition of Fumonisin FB1 production at 50 μM. Growth inhibition of Fusarium proliferatum of these examples varied from 67 to 86% at 50 μM.

| | % inhibition FB1 | % inhibition fungal |
|---|---|---|
| Example No | production at 50 μM | growth at 50 μM |
| 1 | 100 | 67 |
| 2 | 100 | 77 |
| 3 | 100 | 86 |

Production of DON/Acetyl-DON by Fusarium graminearum

Compounds were tested in microtiter plates in DON-inducing liquid media (1g (NH₄)₂HPO₄, 0.2 g MgSO₄x7H₂O, 3g KH₂PO₄, 10g Glycerin, 5 g NaCl and 40 g Sachharose per liter), supplemented with 10% oat extract, containing 0.5% DMSO, inoculated with a concentrated spore suspension of Fusarium graminearum to a final concentration of 2000 spores/ml.

The plate was covered and incubated at high humidity at 28° C. for 7 days.

At start and after 3 days OD measurement at OD620 multiple read per well (square: 3×3) was taken to calculate the growth inhibition.

After 7 days 1 volume of 84/16 acetonitrile/water was added to each well and a sample of the liquid medium was taken and diluted 1:100 in 10% acetonitrile. The amounts of DON and Acetyl-DON of the samples were analysed per HPLC-MS/MS and results were used to calculate inhibition of DON/AcDON production in comparison to a control without compound.

### Examples for Inhibition of DON/AcDON Production

The compounds listed below showed an activity of >80% of inhibition of DON/AcDON at 50 μM.

Growth inhibition of Fusarium graminearum of these examples varied from 41 to 54% at 50 μM.

| | % Inhibition of | % Inhibition of fungal |
|---|---|---|
| Example No | DON/AcDON at 50 μM | growth at 50 μM |
| 1 | 100 | 54 |
| 2 | 100 | 58 |
| 3 | 100 | 41 |

Production of Aflatoxins by Aspergillus parasiticus

Compounds were tested in microtiter plates (96 well black flat and transparent bottom) in Aflatoxin-inducing liquid media (20 g sucrose, yeast extract 4 g, KH₂PO₄ 1 g, and MgSO₄ 7H₂O 0.5 g per liter), supplemented with 20 mM of Cavasol (hydroxypropyl-beta-cyclodextrin) and containing 1% of DMSO. The assay is started by inoculating the medium with a concentrated spore suspension of Aspergillus parasiticus at a final concentration of 1000 spores/ml.

The plate was covered and incubated at 20° C. for 7 days.

After 7 days of culture, OD measurement at OD₆₂₀ₙₘ, with multiple read per well (circle: 4×4) was taken with an Infinite 1000 (Tecan) to calculate the growth inhibition. In the same time bottom fluorescence measurement at Em₃₆₀ₙₘ and Ex₄₂₆ₙₘ with multiple read per well (square: 3×3) was taken to calculate inhibition of aflatoxin formation.

### Examples for Inhibition of Production of Aflatoxins:

The compounds listed below showed an activity of >80% of inhibition of aflatoxins at 50 μM. Growth inhibition of Aspergillus parasiticus of these examples was also 100% at 50 μM.

| | % Inhibition of | % Inhibition of fungal |
|---|---|---|
| Example No | Aflatoxin at 50 μM | growth at 50 μM |
| 1 | 100 | 100 |
| 2 | 100 | 100 |
| 3 | 100 | 100 |

1. A method of reducing mycotoxin contamination in a plant and/or any plant material and/or plant propagation material comprising applying to the plant and/or plant material and/or plant propagation material an effective amount of a compound of formula (I): 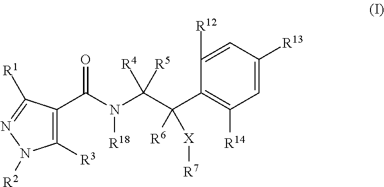
wherein
R¹ is halogenomethyl;
R² is C₁-C₄-alkyl, C₁-C₄-halogenoalkyl, C₁-C₄-alkoxy-C₁-C₄-alkyl or halogenoalkoxy-C₁-C₄-alkyl; and
R³ is hydrogen, halogen, methyl or cyano;
R⁴, R⁵ and R⁶ independently of each other stand for hydrogen, halogen, nitro, C₁-C₆-alkyl, which is unsubstituted or substituted by at least one substituent R⁸, C₃-C₆-cycloalkyl, which is unsubstituted or substituted by at least one substituent R⁸, C₂-C₆-alkenyl, which is unsubstituted or substituted by at least one substituent R⁸, C₂-C₆-alkynyl, which is unsubstituted or substituted by at least one substituent R⁸;
or R⁴ and R⁵ together are a C₂-C₅-alkylene group, which is unsubstituted or substituted by at least C₁-C₆-alkyl group;
X is oxygen, sulfur, —N(R¹⁰)— or —N(R¹¹)—O—:
R¹⁰ and R¹¹ independently of each other stand for hydrogen or C₁-C₆-alkyl;
R⁷ stands for C₁-C₆-alkyl, which is unsubstituted or substituted by at least one substituent R⁹, C₃-C₆-cycloalkyl, which is unsubstituted or substituted by at least one substituent R⁹, C₂-C₆-alkenyl, which is unsubstituted or substituted by at least one substituent R⁹, C₂-C₆-alkynyl, which is unsubstituted or substituted by at least one substituent R⁹;
R¹² stands for halogen, C₁-C₆-halogenoalkoxy, C₁-C₆-halogenoalkylthio, cyano, nitro, —C(R^{a})═N(OR^{b}), C₁-C₆-alkyl, which is unsubstituted or substituted by at least one substituent R¹⁵, C₃-C₆-cycloalkyl, which is unsubstituted or substituted by at least one substituent R¹⁵, C₆-C₁₄-bicycloalkyl, which is unsubstituted or substituted by at least one substituent R¹⁵, C₂-C₆-alkenyl, which is unsubstituted or substituted by at least one substituent R¹⁵, C₂-C₆-alkynyl, which is unsubstituted or substituted by at least one substituent R¹⁵, phenyl, which is unsubstituted or substituted by at least one substituent R¹⁵, phenoxy, which is unsubstituted or substituted by at least one substituent R¹⁵ or pyridinyloxy, which is unsubstituted or substituted by at least one substituent R¹⁵;
R¹³ stands for hydrogen, halogen, C₁-C₆-halogenoalkoxy, C₁-C₆-halogenoalkylthio, cyano, nitro, —C(R^{c})═N(OR^{d}), C₁-C₆-alkyl, which is unsubstituted or substituted by at least one substituent R¹⁶, C₃-C₆-cycloalkyl, which is unsubstituted or substituted by at least one substituent R¹⁶, C₃-C₁₄-bicycloalkyl, which is unsubstituted or substituted by at least one substituent R¹⁶, C₂-C₆-alkenyl, which is unsubstituted or substituted by at least one substituent R¹⁶, C₂-C₆-alkynyl, which is unsubstituted or substituted by at least one substituent R¹⁶, phenyl, which is unsubstituted or substituted by at least one substituent R¹⁶, phenoxy, which is unsubstituted or substituted by at least one substituent R¹⁶ or pyridinyloxy, which is unsubstituted or substituted by at least one substituent R¹⁶;
R¹⁴ stands for hydrogen, halogen, C₁-C₆-halogenoalkoxy, C₁-C₆-halogenoalkylthio, cyano, nitro, —C(R^{e})═N(OR^{f}), C₁-C₆-alkyl, which is unsubstituted or substituted by at least one substituent R¹⁷, C₃-C₆-cycloalkyl, which is unsubstituted or substituted by at least one substituent R¹⁷, C₆-C₁₄-bicycloalkyl, which is unsubstituted or substituted by at least one substituent R¹⁷, C₂-C₆-alkenyl, which is unsubstituted or substituted by at least one substituent R¹⁷, C₂-C₆-alkynyl, which is unsubstituted or substituted by at least one substituent R¹⁷, phenyl, which is unsubstituted or substituted by at least one substituent R¹⁷, phenoxy, which is unsubstituted or substituted by at least one substituent R¹⁷ or pyridinyloxy, which is unsubstituted or substituted by at least one substituent R¹⁷;
each R⁸, R⁹, R¹⁵, R¹⁶ and R¹⁷ is independently of each other halogen, nitro, C₁-C₆-alkoxy, C₁-C₆-halogenoalkoxy, C₁-C₆-alkylthio, C₁-C₆-halogenoalkylthio, C₃-C₆-alkenyloxy, C₃-C₆-alkynyloxy or —C(R^{g})═N(OR^{h});
each R^{a}, R^{c} R^{e} and R^{g} is independently of each other hydrogen or C₁-C₆-alkyl;
each R^{b}, R^{d} R^{f} and R^{h} is independently of each other C₁-C₆-alkyl;
R¹⁸ is hydrogen or C₃-C₇-cycloalkyl; and/or
a tautomer/isomer/enantiomer thereof.

2. The method according to claim 1, wherein R¹⁸ is hydrogen.

3. The method according to claim 1, wherein R¹ is CF₃, CF₂H or CFH₂, optionally CF₂H or CF₃; R² is C₁-C₄-alkyl, optionally methyl; and R³is hydrogen or halogen, optionally hydrogen or chlorine or fluorine and/or optionally R¹ is CF₂H; R² is methyl and R³ is hydrogen.

4. The method according to claim 1, wherein R⁴ is hydrogen or C₁-C₆-alkyl, which is unsubstituted or substituted by at least one substituent R⁸.

5. The method according to claim 1, wherein a compound of formula 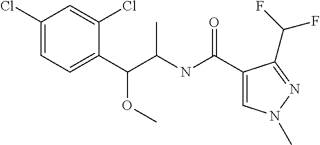 [SMILES: COC(c1ccc(Cl)cc1Cl)C(C)NC(=O)c1cn(C)nc1C(F)F]
is used.

6. The method according to claim 1, wherein a compound of formula 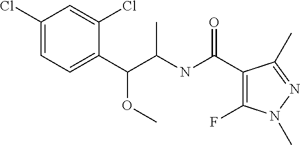 [SMILES: COC(c1ccc(Cl)cc1Cl)C(C)NC(=O)c1c(C)nn(C)c1F]
is used.

7. The method according to claim 1, wherein a compound of formula 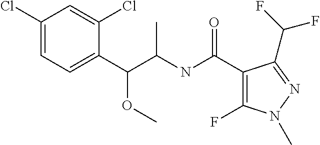 [SMILES: COC(c1ccc(Cl)cc1Cl)C(C)NC(=O)c1c(C(F)F)nn(C)c1F]
is used.

8. The method according to claim 1, wherein said mycotoxins trichothecene mycotoxin.

9. The method according to claim 1, wherein said mycotoxin is deoxynivalenol.

10. A compound as defined as being used in said method of according to claim 1, wherein said compound is capable of reducing mycotoxin contamination in a plant and/or any plant material and/or plant propagation material.

11. A plant and/or plant material and/or plant propagation material treated by the method according to claim 1.
